(19) United States
(12) Patent Application Publication
(10) Pub. No.: US 2020/0080991 A1
(43) Pub. Date: Mar. 12, 2020

(54) SCREENING FOR AGENTS THAT TARGET THE ACTIN CYTOSKELETON USING C. ELEGANS EXPOSED TO HEAT SHOCK

(71) Applicant: Rutgers, The State University of New Jersey, New Brunswick, NJ (US)

(72) Inventors: Federico Sesti, Somerville, NJ (US); Rahul Patel, Monmouth Jct., NJ (US)

(73) Assignee: Rutgers, The State University of New Jersey, New Brunswick, NJ (US)

(21) Appl. No.: 16/468,239

(22) PCT Filed: Jan. 19, 2018
(86) PCT No.: PCT/US2018/014330

§ 371 Date: Jun. 10, 2019

Related U.S. Application Data
(60) Provisional application No. 62/448,435, filed on Jan. 20, 2017.

(51) Int. Cl. G01N 33/50; A01K 67/033

(52) U.S. Cl. CPC G01N 33/5085; A01K 67/0336; A01K 2217/075; A01K 2267/0393; A01K 2227/703

(57) ABSTRACT

Provided herein are screening methods that utilize transgenic nematodes exposed to heat shock conditions, such as agents that increase stability of F-actin. The transgenic nematodes used can be in a wild-type background, functionally deleted for OSG-1, or express human ARHGEF10 in an OSG-1 background. Such transgenic nematodes also express a fluorescent protein, such as GFP. Such methods are in some examples high throughput and automated. Also provided are recombinant nematodes and kits that can be used with such methods.

### CROSS-REFERENCE TO RELATED APPLICATION

This application claims priority to U.S. Provisional Application No. 62/448,435 filed Jan. 20, 2017, herein incorporated by reference in its entirety.

### ACKNOWLEDGMENT OF GOVERNMENT SUPPORT

This invention was made with government support under 0842708, 1026958 and 145667 awarded by The National Science Foundation. The government has certain rights in the invention.

### FIELD

This application relates to methods that utilize transgenic nematodes genetically engineered to expresses a fluorescent protein (e.g., in a normal/wild type background, nematodes that are functionally deleted for OSG-1 (ΔOSG-1), or nematodes that express human ARHGEF10 in a ΔOSG-1 background), which have been exposed to heat shock conditions, for screening to identify agents that target the actin cytoskeleton. Such methods are in some examples high throughput.

### BACKGROUND

Both in vitro and in vivo pharmaceutical screenings are a central need of the pharmaceutical industry. Phenotypic screening technologies are relevant to basic and translational research to identify novel pathways and proprietary druggable targets. Typically, the search for a new drug starts with high-throughput screening (HTS) using isolated enzymes, cellular or bacterial assays. Once potential hits are identified and characterized, pharmacodynamic efficacy is established using rodents or other mammals. However, bacteria and unicellular organisms are highly isolated systems and may not accurately reflect the perturbation of the pathway of interest in a complete organism, such as a higher eukaryote. For this reason their screening efficacy is limited, because often times a compound that works in bacteria or cellular assays fails to be effective in mice or other mammals. But screening in mice, is expensive and slow, and therefore, all potential hits identified in a bacterial or cellular screening assay are not usually tested in an animal model. Therefore, there is a need for intermediary, cost-effective assays that allows for additional compound testing in an intact organism prior to further development.

### SUMMARY

Provided herein are methods for identifying an agent that targets actin, such as agents that increase the stability of F-actin. Such agents can be used to treat a disease associated with F-actin instability, such as a neurological disorder. The disclosed methods assesses the ability of a test compound to alter (e.g., increase or decrease), the response of a nematode (such as Caenorhabditis elegans) to a thermal insult (heat shock, HS). In some examples, such methods include exposing the nematode to one or more test compounds during the early stages of nematode development. One skilled in the art will appreciate the multiple test agents can be screened simultaneously or contemporaneously.

In some examples, the method includes incubating a test agent with a transgenic nematode genetically engineered to expresses a fluorescent protein (e.g., in the pharynx) in a normal (e.g., C. elegans N2 (Bristols strain)) background. In other examples, the method includes incubating a test agent with a transgenic first nematode that is functionally deleted for OSG-1 (referred to herein as ΔOSG-1) and expresses a fluorescent protein and with a transgenic second nematode that expresses a functional mammalian ARHGEF10 protein (such as a human ARHGEF10 protein) and a fluorescent protein in a ΔOSG-1 background, wherein the first and the second nematodes are incubated separately in a culture/growth medium.

The culture/growth medium can be a liquid medium (such as S buffer), or a solid medium (such as nematode growth medium (NGM)). The nematodes can be incubated in the culture medium for at least 24 hours, at least 48 hours, at least 72 hours, or at least 96 hours, for example at 15° C. to 25° C., such as 15° C. to 22° C., such as 20° C.

The survival rate of the nematodes are measured or determined prior to heat shock (HS) conditions. The HS simultaneously triggers numerous signaling cascades. The nematodes are subsequently exposed to heat shock conditions, such as incubation at 35° C. to 39° C. for at least 1 hour, at least 2 hours, or at least 4 hours. Following the heat shocking (such as at least 12 hours, at least 18 hours or at least 24 hours later), the survival rate of the nematodes are measured or determined. This allows for the determination of a test agent's ability to promote survival of the nematode.

If using transgenic nematode genetically engineered to expresses a fluorescent protein (e.g., in the pharynx) in a normal (e.g., C. elegans N2 (Bristols strain)) background, a test agent that alters the ratio of survival rate of the nematode in the test agent compared to the same ratio in the absence of the test agent, is an agent that can be selected for further analysis. In some examples, such agents are ones that target the actin cytoskeleton (e.g., stabilizes F-actin), but may target other proteins. Thus, if Na is the number of the worms surviving the HS in the absence of the compound, and Nb is the number of worms surviving the HS in the presence of the compound, the survival rate for absence of the compound is

Na

Total number of worms

and the survival rate for presence of the compound is:

Nb

Total number of worms

If the values for these two ratios differs (e.g., the value of Nb/Total number of worms>Na/Total number of worms), the agent can be selected, for example further analysis in a mammal, such as a mouse. In some examples, such agents are ones that target the actin cytoskeleton (e.g., stabilizes f-actin), but may target other proteins.

If using a first nematode that is ΔOSG-1 and expresses a fluorescent protein and a transgenic second nematode that expresses a functional mammalian ARHGEF10 protein and a fluorescent protein in a ΔOSG-1 background, a test agent that alters the ratio of survival rate of the transgenic second nematode (ARHGEF10) in the test agent to survival of the transgenic first nematode (ΔOSG-1) in the test agent as compared to the same ratio in the absence of the test agent is an agent, is one that targets actin (e.g., stabilizes f-actin). Such agents that target the actin cytoskeleton can be selected, for example further analysis in a mammal, such as a mouse.

In some examples determining survival of the nematodes includes measuring fluorescence (such as fluorescence intensity) generated from the fluorescent protein from the nematodes (such as the first and the second nematodes). In some examples, such a step includes counting individual nematodes.

Heat shock methods, which can be used in the methods for testing agents for their ability to target (e.g., stabilize) actin, are provided. Such methods can include incubating a test organism (such as a nematode) at a temperature of about 30° C. for 1 to 60 minutes). The temperature is then increased to a temperature of 31° C. to 39° C. (such as 31° C., 32° C., 33° C., 34° C., 35° C., 36° C., 37° C., 38° C., or 39° C.±0.5 or ±0.1° C.) in temperature increments of 0.1° C. to 1° C. (such as increments of 0.2° C. or 0.5° C.), wherein each temperature increment is performed for 1 to 45 minutes (such as 10 to 45 minutes). The test organism is then maintained the temperature of 31° C. to 39° C. (such as 31° C., 32° C., 33° C., 34° C., 35° C., 36° C., 37° C., 38° C., or 39° C. ±0.5 or ±0.1° C.) for 5 minutes to 120 minutes (such as 5 to 30 minutes, 5 to 60 minutes, or 10 to 30 minutes).

Also provided are isolated transgenic nematodes that expresses a functional mammalian ARHGEF10 protein and a fluorescent protein in a ΔOSG-1 background. Such transgenic nematodes can be used in the methods and kits provided herein.

Also provided are kits that include (1) a transgenic first nematode that is functionally deleted for OSG-1 and expresses a fluorescent protein; and (2) a transgenic nematode that expresses a functional mammalian ARHGEF10 protein in a ΔOSG-1 background and a fluorescent protein. In some examples, such transgenic nematodes can be in separate containers (such as a glass or plastic vial). Such kits can include additional agents, such as multi-well plate(s), culture medium (such as a solid or liquid medium), positive control(s), negative control(s), E. coli OP50, and combinations thereof.

The foregoing and other objects and features of the disclosure will become more apparent from the following detailed description, which proceeds with reference to the accompanying figures.

### BRIEF DESCRIPTION OF THE DRAWINGS

FIG. 1 is a schematic drawing providing an overview of the method.

FIG. 2 is a schematic drawing providing an overview of the assay results when using nematodes that express ARHGEF10, or are functionally deleted for OSG-1, in the presence of a test compound.

FIG. 3 is a graph showing an exemplary gradient heat shock protocol.

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “sequence listing.txt” (−72 kb), which was created on Jan. 12, 2018, and which is incorporated by reference herein.

SEQ ID NOS: 1 and 2 provide an exemplary human ARHGEF10 nucleic acid and protein sequences, respectively. Source: GenBank Accession Nos: BC112926.1 and AAI12927.1. The coding sequence is nucleotides 174-4094 of SEQ ID NO: 1.

SEQ ID NOS: 3 and 4 provide an exemplary C. elegans ORG-1 nucleic acid and protein sequences, respectively. Source: GenBank Accession Nos: NM_065771.5 and Q21653.4.

SEQ ID NO: 5 provides an exemplary C. elegans OSG-1 promoter sequence.

SEQ ID NO: 6 provides an exemplary myo-2 promoter sequence (nt 1-1374) operably linked to a GFP coding sequence.

SEQ ID NOS: 7 and 8 provide primer sequences.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a protein” includes single or plural proteins and is considered equivalent to the phrase “comprising at least one protein.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Jan. 20, 2017. All references and GenBank® Accession numbers cited herein are incorporated by reference in their entireties.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

Administration: To provide or give a subject (such as a laboratory mammal) an agent, such as a test agent identified using the methods disclosed herein, by any effective route. Exemplary routes of administration include, but are not limited to, oral, injection (such as subcutaneous, intramuscular, intradermal, intraperitoneal, intravenous, intrathecal, and intratumoral), sublingual, rectal, transdermal, intranasal, vaginal and inhalation routes.

Actin: A family of globular multi-functional proteins that form microfilaments, which is found in most eukaryotic cells. Actin is the monomeric subunit of two types of filaments in cells: microfilaments, one of the three major components of the cytoskeleton, and thin filaments, part of the contractile apparatus in muscle cells. It can be present as either a free monomer called G-actin (globular) or as part of a linear polymer microfilament called F-actin (filamentous), both of which are essential for cellular functions such as the mobility and contraction of cells during cell division. Many diseases are caused by mutations in alleles of the genes that regulate the production of actin or of its associated proteins. The production of actin is also key to the process of infection by some pathogenic microorganisms. Mutations in the different genes that regulate actin production in humans can cause muscular diseases, variations in the size and function of the heart as well as deafness. The make-up of the cytoskeleton is also related to the pathogenicity of intracellular bacteria and viruses, particularly in the processes related to evading the actions of the immune system. Thus, the agents identified using the disclosed methods can be used to treat cytoskeletal disorders that results from F-actin instability, such as congenital myopathies (e.g., those in skeletal muscle), Type 1R dilated cardiomyopathy and Type 11 hypertrophic cardiomyopathy in the heart, juvenile dystonia, infection mechanisms, nervous system malformations (such as a neurodegenerative disease, e.g., bipolar disease, Huntington's disease, Alzheimer's disease, temporal lobe epilepsy, and acquired immunodeficiency syndrome-related dementia), and tumor invasion (such as a cancer of the lung, liver, breast, prostate, kidney, colon, or skin).

ARHGEF10 (Rho guanine nucleotide exchange (GEF)factor 10): e.g., OMIM 608136. Includes ARHGEF10 nucleic acid molecules and proteins. A guanine nucleotide exchange factor (GEF) for RhoA with proposed roles in various diseases. ARHGEF10 is a member of the family of Rho guanine nucleotide exchange factors (GEFs), which are implicated in neural morphogenesis and connectivity and regulate the activity of small Rho GTPases by catalyzing the exchange of bound GDP by GTP.

ARHGEF10 sequences are publically available, for example from GenBank® sequence database (e.g., Accession Nos. AAI12927.1, AAH59212.1, and XP 006253465.1 provide exemplary ARHGEF10 protein sequences, while Accession Nos. BC112926.1, XM_006253403.3 and XM_006508773.3 provide exemplary ARHGEF10 nucleic acid sequences). One of ordinary skill in the art can identify additional ARHGEF10 nucleic acid and protein sequences, including ARHGEF10 variants. In some examples, a mammalian ARHGEF10 expressed by a recombinant nematode has at least 90%, at least 92%, at least 95%, at least 96%, at least 97%, at least 98%, or at least 99% sequence identity to SEQ ID NO: 1 or 2, wherein such a variant retains ARHGEF10 biological activity.

Contact: To bring one agent into close proximity to another agent, thereby permitting the agents to interact. For example, a test agent can be incubated with a transgenic nematode disclosed herein, thereby permitting identification of test agents that target the actin cytoskeleton.

Detect: To determine if an agent or organism is present or absent, for example to measure an amount (qualitatively or quantitatively) of agent present or number of organisms present. In some examples this can further include quantification. For example, use of the disclosed methods include detecting fluorescent protein produced by a transgenic nematode expressing the fluorescent protein, thereby permitting detection of the nematode, and determining with the nematode is alive or dead. In some examples, fluorescent protein is detected by flow cytometry or fluorescence microscopy. Detection can be in bulk, so that a macroscopic number of molecules can be observed contemporaneously or simultaneously. Detection can also include detection of single events, such as a single nematode.

Fluorescent protein: A protein, which when excited by exposure to a particular wavelength of light, emits light (fluoresces), for example at a different wavelength. Specific examples of fluorescent proteins that can be used in the disclosed methods and recombinant organisms include but are not limited to: green fluorescent protein (GFP), blue fluorescent protein (BFP), cyan fluorescent protein (CFP), enhanced GFP (EGFP), red florescent protein (RPE, r-phycoerythrin), dsRed, red fluorescent protein (mCherry), yellow fluorescent protein (YFP). mCherry and many red fluorescent proteins derive from a protein isolated from Discosoma sp., while fluorescent proteins in the green range are often variants of GFP from Aequorea victoria.

Culture Medium: A liquid or solid (such as a gel) that permits and supports the growth of microorganisms, such as nematodes. Includes nutrient broths and solid media that contain components necessary for nematode growth and replication, such as water, a carbon source (such as glucose) and salts. Such media can include other agents, such as agar, E. coli (e.g., as a food source) vitamins and amino acids.

Heat Shock (HS): The process of subjecting a nematode to a higher temperature than that of the ideal or usual growth temperature for the nematode. For example, if the ideal growth temperature for the nematode is less than about 25° C., such as 15° C. to 25° C., such as 20° C. to 22° C., such as 22° C., then heat shock can include subjecting the nematode to a higher temperature, such as at least 30° C., at least 35° C., such as 31° C. to 39° C., or 35° C. to 39° C., such as 37° C.

Isolated: An “isolated” biological component (such as a nematode) has been substantially separated, produced apart from, or purified away from other components in the sample in which the nematode occurs, such as, other cells, nucleic acids, and proteins. Nematodes which have been “isolated” thus include cells purified by standard purification methods, such as filtration and centrifugation. The nematodes need not be 100% pure, but includes nematodes where at least 50% of the other materials in the sample have been separated away from the nematodes, such as at least 75%, at least 80%, at least 90%, or at least 95% of the other materials in the sample have been separated away from the nematodes in the sample.

Mammal: This term includes both human and non-human mammals. Similarly, the term “subject” includes both human and veterinary subjects (such as cats, dogs, cows, and pigs) and rodents (such as mice and rats).

Nematode: Any nematode of the class Secernentea useful for genetic research, such as member of the genus Caenorhabditis. For example, C. elegans, C. briggsae, and C. vulgaris can be used for the disclosed assays. In one example, the nematode is C. elegans.

OSG-1: Includes OSG-1 nucleic acid molecules and proteins. A guanine nucleotide exchange factor that controls actin dynamics in C. elegans. OSG-1 confers functional aging by dysregulating Rho GTPases activities in C. elegans. Loss of OSG-1 gene function lessenes loss of function (chemotaxis) in ASE sensory neurons subjected to conditions of oxidative stress generated during natural aging, by oxidative challenges or genetic mutations (see Duan and Sesti, Genetics, 199:487-96, 2015). Loss of OSG-1 gene function also results in significantly lower thermotolerance (about 90% mortality) as compared to wild type worms (about 50% mortality) when subjected to hyperthermia (e.g., heat shock).

OSG-1 sequences are publically available, for example from GenBank® sequence database (e.g., Accession No. Q21653.4 provides an exemplary OSG-1 protein sequence, while Accession No. NM 065771.5 provides an exemplary OSG-1 nucleic acid sequence). One of ordinary skill in the art can identify additional OSG-1 nucleic acid and protein sequences, including OSG-1 variants. In some examples, an OSG-1 functionally deleted or knocked out in a recombinant nematode has at least 90%, at least 92%, at least 95%, at least 96%, at least 97%, at least 98%, or at least 99% sequence identity to SEQ ID NO: 3 or 4.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as a human ARHGEF10 coding sequence or a fluorescent protein coding sequence). Generally, operably linked DNA sequences are contiguous and, where necessary to join two protein coding regions, in the same reading frame.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15^{th} Edition (1975), describes compositions and formulations suitable for pharmaceutical delivery of a test agent.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. Examples of promoters that can be used include constitutive promoters, and inducible promoters.

Recombinant: A recombinant nucleic acid molecule or protein is one that has a sequence that is not naturally occurring (e.g., a human ARHGEF10 coding sequence operably linked to a C. elegans promoter) or has a sequence that is made by an artificial combination of two otherwise separated segments of sequence. This artificial combination can be accomplished by routine methods, such as chemical synthesis or by the artificial manipulation of isolated segments of nucleic acids, such as by genetic engineering techniques. Similarly, a recombinant or transgenic organism is one that contains a recombinant nucleic acid molecule and expresses a recombinant protein, such as an exogenous (e.g., non-native) nucleic acid molecule/protein.

Sequence identity of amino acid sequences: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn, and tblastx. A description of how to determine sequence identity using this program is available on the NCBI website on the internet.

Variants of the ARHGEF10 and OSG-1 proteins and coding sequences disclosed herein are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Thus, a variant ARHGEF10 or OSG-1 coding sequence can share at least 80%, at least 85%, at least 90%, at least 91%, at least 92%, at least 95%, at least 96%, at least 97%, at least 98% or at least 99% sequence identity to SEQ ID NO: 1 or 3, respectively. Similarly, a variant ARHGEF10 or OSG-1 protein can share at least 80%, at least 85%, at least 90%, at least 91%, at least 92%, at least 95%, at least 96%, at least 97%, at least 98% or at least 99% sequence identity to SEQ ID NO: 2 or 4, respectively.

Subject: Any mammal, such as humans, non-human primates, pigs, sheep, cows, dogs, cats, rodents and the like which is to be the recipient of the particular treatment, such as treatment with a test agent identified using the methods provided herein. In two non-limiting examples, a subject is a human subject or a murine subject.

Transduced and Transformed: The process of introducing a nucleic acid molecule into a cell, such as the cell of a nematode. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known, such as: chemical methods (e.g., calcium-phosphate transfection), physical methods (e.g., electroporation, microinjection, particle bombardment), fusion (e.g., liposomes), receptor-mediated endocytosis. For C. elegans, microinjection and particle bombardment are typically used.

Transgene: An exogenous gene or other nucleic acid molecule, for example exogenous to the cell or organism into which the gene or other nucleic acid molecule is introduced. In one example, a transgene includes a mammalian ARHGEF10 coding sequence, which is exogenous relative to a nematode. In one example, a transgene includes a fluorescent protein coding sequence, which is exogenous relative to a nematode.

Vector: A nucleic acid molecule as introduced into a host cell, thereby producing a transformed host cell. A vector may include nucleic acid sequences that permit it to replicate in the host cell, such as an origin of replication. A vector may also include one or more coding sequences and/or selectable marker genes and other genetic elements known in the art. A vector can transduce, transform, or infect a cell, thereby causing the cell to express nucleic acids and/or proteins other than those native to the cell. A vector optionally includes materials to aid in achieving entry of the nucleic acid into the cell, such as a viral particle, liposome, protein coating, or the like.

### Overview

Disclosed herein are new methods for identifying therapeutic compounds that target the actin cytoskeleton, such as agents that increase the stability of F-actin (for example in response to heat shock conditions). In some examples, such agents directly bind to actin or directly or indirectly alter F-actin polymerization (e.g., by altering F-actin, actin binding proteins, or signaling pathways that regulate actin polymerization, such as for example Rho signaling). In some examples, agents identified using the disclosed methods can be used in therapeutic amounts to treat a disease associated with F-actin instability, such as a neurodegenerative disease (such as bipolar disease, Huntington's disease, Alzheimer's disease, temporal lobe epilepsy, and acquired immunodeficiency syndrome-related dementia), a cardiovascular disease, a congenital myopathy (such as nemaline myopathy (NM), actin myopathy (AM), and intranuclear rod myopathy (IRM)), or cancer (such as a cancer of the lung, liver, breast, prostate, kidney, colon, or skin).

Nematodes, such as C. elegans, that express a fluorescent protein, such as GFP, are used in the methods to score for survival in the presence of test compounds. Most currently available screening assays require screening in bacteria, which do not confirm with eukaryotes. However, prior to the present methods, the use of nematodes in a high throughput automated assay was difficult, as the worms lay eggs, and need to be feed (e.g., grown in media). However, because the disclosed methods can be completed in a week, the production of eggs and larvae during this time are not sufficient to interfere with the method in this short time frame. In addition, the assay can be performed in the presence of growth/culture media, for example by using multi-well plates. This also allows for automation. Screening in a live eukaryotic system as provided herein can produce insights on the mechanism of action as well as which can be beneficial in a human.

The disclosed assays offer high-throughput screenings operating in the segment between in vitro assays and rodent studies. The screening methods allow for the large scale capability of in vitro assays, at a low cost and low maintenance in a more physiological appropriate organism due to the intricate and well defined systems the worm carriers. In addition, the testing is done on human genes, a feature that allows for the effective study and targeting of human genes in a physiologically relevant animal model. The disclosed binary screening allows for testing hundreds or thousands of compounds per week.

The disclosed methods provide a statistical probability that the test compound with be efficacious in vertebrate organisms, such as Homo sapiens. For each tested compound there are three possibilities. First are test compounds worsen or reduce the response of the nematodes (that is, the survival following exposure to HS in the presence of the test compound is reduced as compared to the absence of the compound). This type of molecule is not recommended to progress to vertebrate testing (e.g., such compounds are not selected for further study). Second are test compounds that do not affect the response of the nematodes (that is, there is no change in the survival following exposure to HS in the presence of the test compound). Here, although the test compound is not toxic, it may not interact with the signaling pathways elicited by the HS. The predictive power of such compounds is null (that is, it cannot be predicted whether such compounds will be efficacious in a vertebrate). Third are test compounds that moderate the response of the nematodes (that is, the survival following exposure to HS in the presence of the test compound is increased as compared to the absence of the compound). Such compounds are expected to have an increased probability of being efficacious in a vertebrate, such as a mammal, such as a human.

The methods can expose a test compound to multiple signaling pathways in vivo. The HS is given to simultaneously trigger numerous signaling cascades. Thus, the methods allow for an evaluation of test agents that may provide therapeutic treatment for a number of conditions including exposure to environmental stresses or pathologic conditions, such as ischemia, inflammation, tissue damage, and infection. In this paradigm, the worms are exposed to the test compound prior to HS and subsequently analyzed for efficacy to promote survival. Compounds that increase survival compared to control can be selected for further investigation.

The methods use transgenic nematodes, such as C. elegans, that express a fluorescent protein, such as GFP. In one example, the transgenic nematodes express the fluorescent protein in a normal or wild-type (WT) background, such as an N2 C. elegans. Subject to hyperthermia, these transgenic worms exhibit significantly lower thermotolerance (about 50% mortality). Thus, methods are provided herein wherein the transgenic worms are exposed to one or more test agents prior to heat shock, heat shocked, and the worms subsequently analyzed to determine the ability of the test agents to promote survival. Efficacious compounds can be identified by comparing the results (e.g., by comparing fluorescence intensity measured or comparing the number of fluorescent and non-fluorescent worms counted) before/after heat shock in the presence and absence of the test compound. Agents that improve survival following HS have higher likelihood to be efficacious in higher organisms (e.g., vertebrates or mammals) and therefore can be selected for further screening. For example, prior to heat shock all worms should be alive in both the presence and absence of the test agent (if not, this indicates the test agent is toxic), as indicated by detection of the fluorescent protein. The viability of the transgenic nematodes following HS (+/−test agent) is compared to the viability of the transgenic nematodes prior to the heat shock (+/−test agent). If in the presence of the test agent an increase in the viability of the worms is observed after the HS, the test agent is one that targets actin cytoskeleton (e.g., stabilizes F-actin), and can be selected for further study.

In another example, the transgenic nematodes express the fluorescent protein and further express a mammalian (e.g., human) ARHGEF10 in a ΔOSG-1 background, or are functionally deleted for the C. elegans ortholog OSG-1 (ΔOSG-1). As the actin cytoskeleton is highly conserved, a transgenic C. elegans gain of function containing human ARHGEF10 can be used in the assays provided herein. This gene, and its C. elegans ortholog OSG-1, modulate the integrity and dynamics of the actin cytoskeleton. Subject to hyperthermia, OSG-1 knockout (KO) worms exhibit significantly lower thermotolerance (about 90% mortality) as compared to wild type worms (about 50% mortality). When ARHGEF10 is introduced into OSG-1 KO background (ΔOSG-1), worms regain wild type thermotolerance (50% mortality). Thus, methods are provided herein wherein both groups of worms are exposed to one or more test agents prior to heat shock and the worms subsequently analyzed to determine the ability of the test agents for efficacy to promote survival. Only compounds that target the cytoskeleton can differentially change the mortality rates of OSG-1KO compared to the ARHGEF10 worms.

The heat shock response allows for examination in an animal model in a number of exposures, such as environmental stresses or pathologic conditions, like ischemia, inflammation, tissue damage, and infection. Thermal stress produces large trafficking of proteins within the cell primarily due to damaged proteins needing replacement and/or repair. Heat shock is like a major storm: after the storm, many emergency responses are activated (rescue efforts, reconstruction of the damaged area, etc.). Similarly, in response to a heat shock, numerous proteins must be quickly displaced in different parts of the cell to mount effective rescue and repair. But cargo does not freely move throughout the cytoplasm. Proteins and macromolecules must move through cellular highways: the actin filaments. This is why during the heat shock the actin cytoskeleton plays a crucial role.

The disclosed methods offer a novel method to screen the efficacy of therapeutic compounds targeting the actin cytoskeleton and its associated genes. In this paradigm, the two groups of worms are exposed to the compound of interest prior to thermal insult and subsequently analyzed for efficacy to promote survival. One of the strengths of this method is the binary nature of the results. Because the mortality of OSG-1 KO worms is significantly higher than wild type, only compounds that target the actin cytoskeleton pathway can differentially change the mortality rates of the KO compared to the transgenic worm. Compounds that are efficacious but affect the mortality rates of the KO and the transgenic worm to the same extent do not impinge on the actin cytoskeleton pathway. As such, they may act on pathways and mechanisms that are not conserved in humans and therefore can be considered for additional investigation.

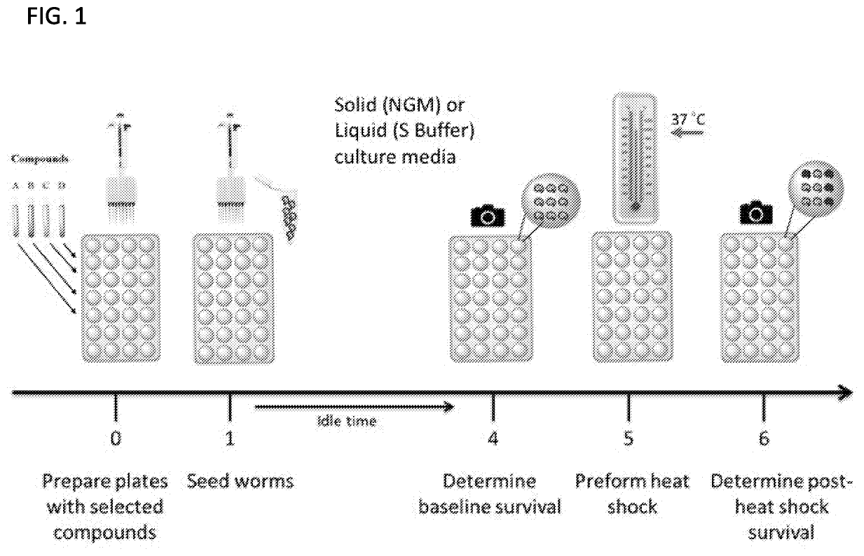

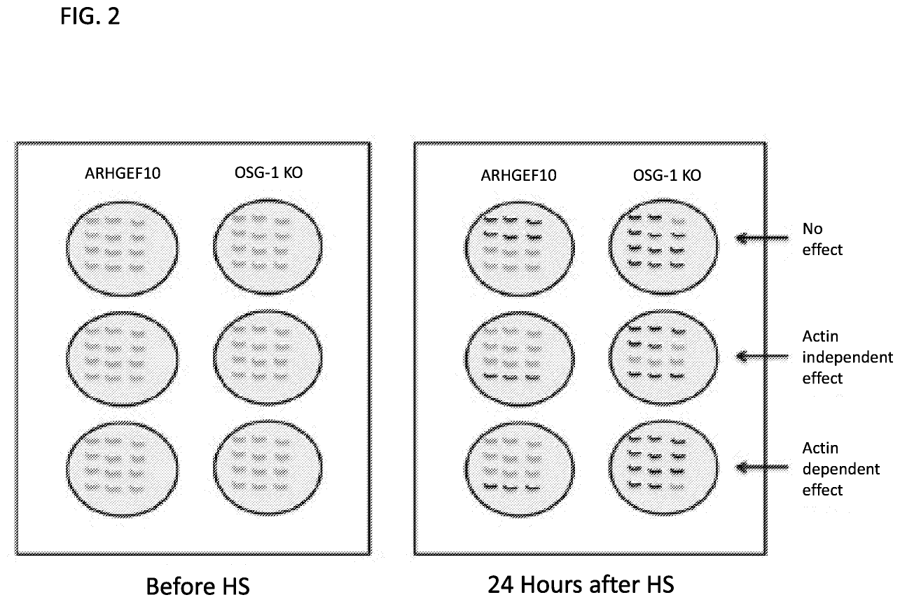

A summary of one embodiment of the method is provided in FIG. 1. The procedure is time efficient and can be completely automated. As shown in FIG. 1, multiwell plates containing one or more test agents (e.g., compounds A, B, C and D) and culture media (liquid or solid) are provided (day 0). Negative controls can include no test compound. Age synchronized L1 larvae (for example N2 larvae, or ΔOSG-1 worms and ARHGEF10 in a ΔOSG-1 background worms that are in different wells) in liquid or solid media are transferred into the multiwell plates (e.g., 25±2 larvae/well for a 96 well plate), each containing culture media and a test compound(s) (or control) (day 1). Worms are allowed grow for 3 to 5 days at their normal growth temperature (e.g., 20° C.-22° C.) without any intervention (idle time, days 1 to 4). At day 5 the plates containing now fully developed, adult worms, are subjected to a heat shock (e.g., 37° C. for 2 or 4 hours, 4 hours for solid media, 2 hours for liquid media) and returned to the incubator. 24 hours later (day 6) surviving worms are counted, for example by detecting fluorescence. The transgenic worms are engineered to express a fluorescent protein (such as GFP) in the pharynx or through most of the body. Only live worms express detectable fluorescent protein whereas dead ones do not (FIG. 2). Assessment of survival can be achieved by measuring fluorescence generated from the recombinantly expressed fluorescent protein, for example by counting individual fluorescent worms (e.g., in a 6- or 12-well plate) or by measuring fluorescence intensity (e.g., in a 96- or 384-well plate) for each condition. Plates are imaged, for example with a normal Nomarski microscope equipped with a digital camera, under appropriate filters and light, before and after the heat shock.

FIG. 2 shows an exemplary ass where ΔOSG-1 worms and ARHGEF10 in a ΔOSG-1 background worms are used in the methods (where in each type of worm is in a separate well). As shown in FIG. 2, efficacious compounds are identified by comparing the results (e.g., by comparing fluorescence intensity measured or comparing the number of fluorescent and non-fluorescent worms counted) before/after heat shock. The left panel in FIG. 2 shows that prior to heat shock (HS) all 12 worms in each well are alive, as indicated by detection of the fluorescent protein. The right panel in FIG. 2 shows the viability of the transgenic nematodes following HS. The top two wells show that following HS, under control conditions (e.g., no test agent), ARHGEF10/fluorescent protein worms have a 3-fold higher (2.73±0.11) survival rate than ΔOSG-1/fluorescent protein worms (referred to in FIG. 2 as OSG-1 KO) (e.g., 6 live 6 dead for ARHGEF10 worms, 1 live 11 dead for ΔOSG-1 worms). Thus, any test agent that changes this survival rate (e.g., increases or decreases) in a statistically significant fashion, is an agent that targets the actin cytoskeleton. For example, the bottom two wells in the right panel of FIG. 2 show that agents that increase the survival of ARHGEF10/fluorescent protein worms (e.g., 9 live 3 dead for ARHGEF10 worms), but not the survival of ΔOSG-1 worms (e.g., 1 live 11 dead for OSG-1 KO worms) indicates that the test agent is acting in an actin-dependent manner. However, agents that change the survival rate of both ARHGEF10/fluorescent protein and ΔOSG-1/fluorescent protein worms to the same extent (so that the ratio between the survival rate of ARHGEF10/fluorescent protein and ΔOSG-1/fluorescent protein worms remains the same, e.g., 9 live 3 dead for ARHGEF10 worms, 4 live 8 dead for ΔOSG-1 worms) indicates that the test agent is acting in an actin-independent manner (see the middle two wells in the right panel of FIG. 2).

### Recombinant Nematodes

Provided herein are recombinant or transgenic nematodes, such as C. elegans, C. briggsae, or C. vulgaris. In a specific example, the recombinant or transgenic nematode is C. elegans. These recombinant/transgenic nematodes can be used in the methods provided herein, and can be a part of the kits provided herein.

Exemplary methods to transform nematodes include DNA transformation (for example by gene bombardment) and microinjection. Such techniques typically utilize co-transformation with a scoreable or selectable marker gene, such as a mutant collagen (rol-6(su1006)) that induces a dominant “roller” phenotype, where animals corkscrew around in circles, an unc-22 anti-sense plasmid (dominant twitcher phenotype), a mutant-rescuing plasmid, or a fluorescent protein (such as GFP).

### PMyo-2:GFP

Provided are recombinant nematodes (N2 background) that express GFP protein in the pharynx under the myo-2 promoter. One skilled in the art will appreciate that other fluorescent proteins and different promoters can be used in a wild-type background.

### ΔOSG-1

Provided are recombinant nematodes that are (1) functionally deleted for OSG-1 (e.g., does not express functional OSG-1 protein, and thus is genetically inactivated for OSG-1) and (2) express a fluorescent protein. Any method for genetic inactivation of OSG-1 (referred to herein as AOSG-1) can be used, as long as expression of the OSG-1 protein is significantly reduced or eliminated (such as a reduction of at least 90%, at least 95%, at least 99%, at least 99.5%, or 100%), or the function of the expressed OSG-1 protein is significantly reduced or eliminated (such as a reduction of at least 90%, at least 95%, at least 99%, at least 99.5%, or 100%). In particular examples, the OSG-1 gene is genetically inactivated by complete or partial deletion mutation or by insertional mutation. In some examples, genetic inactivation need not be 100% genetic inactivation. In some embodiments, OSG-1 genetic inactivation refers to at least 50%, at least 60%, at least 70%, at least 80%, at least 90%, least 95%, at least 98%, at least 99% or at least 99.9% OSG-1 gene or protein inactivation. The term “reduced” or “decreased” as used herein with respect to a nematode and a particular gene or protein activity refers to a lower level of activity than that measured in a comparable organism of the same species. For example, a particular nematode lacking OSG-1 activity has reduced OSG-1 activity if a comparable nematode not having an OSG-1 genetic inactivation has detectable OSG-1 activity.

OSG-1 sequences are disclosed herein and others are publicly available, for example from GenBank or EMBL. In some examples, the OSG-1 gene functionally deleted encodes a protein having at least 80%, at least 90%, at least 91%, at least 92%, at least 93%, at least 94%, at least 95%, at least 96%, at least 97%, at least 98%, at least 99%, or 100% sequence identity to SEQ ID NO: 4. In some examples, the OSG-1 gene functionally deleted has at least 80%, at least 90%, at least 91%, at least 92%, at least 93%, at least 94%, at least 95%, at least 96%, at least 97%, at least 98%, at least 99%, or 100% sequence identity to SEQ ID NO: 3.

The inactivation of OSG-1 results in significantly lower thermotolerance (about 90% mortality) as compared to wild type worms (about 50% mortality), when exposed to heat shock conditions (see FIG. 2).

A. Methods of Functionally Deleting Genes

As used herein, an “inactivated” or “functionally deleted” OSG-1 gene means that the OSG-1 gene has been mutated, for example by insertion, deletion, or substitution (or combinations thereof) of one or more nucleotides such that the mutation substantially reduces (and in some cases abolishes) expression or biological activity of the encoded OSG-1 gene product. The mutation can act through affecting transcription or translation of the gene or its mRNA, or the mutation can affect the OSG-1 peptide product itself in such a way as to render it substantially inactive.

Genetic inactivation of OSG-1 (which in some examples is also referred to as functional deletion) can be performed using any conventional method. In one example, a nematode is transformed with a vector which has the effect of down-regulating or otherwise inactivating an OSG-1 gene. This can be done by mutating control elements such as promoters and the like which control gene expression, by mutating the coding region of the gene so that any protein expressed is substantially inactive, or by deleting the OSG-1 gene entirely. For example, an OSG-1 gene can be functionally deleted by complete or partial deletion mutation (for example by deleting a portion of the coding region of the gene) or by insertional mutation (for example by inserting a sequence of nucleotides into the coding region of the gene, such as a sequence of about 1-5000 nucleotides). Thus, the disclosure in some examples provides transformed nematodes that include at least one exogenous nucleic acid molecule which genetically inactivates an OSG-1 gene (such as a nucleic acid sequence encoding SEQ ID NO: 4). In one example, such a transformed nematode has significantly lower thermotolerance (about 90% mortality) as compared to wild type worms with a functional OSG-1 gene (about 50% mortality), when exposed to heat shock conditions. In particular examples, an insertional mutation includes introduction of a sequence that is in multiples of three bases (e.g., a sequence of 3, 9, 12, or 15 nucleotides) to reduce the possibility that the insertion will be polar on downstream genes. For example, insertion or deletion of even a single nucleotide that causes a frame shift in the open reading frame, which in turn can cause premature termination of the encoded OSG-1 polypeptide or expression of a substantially inactive polypeptide. Mutations can also be generated through insertion of foreign gene sequences, for example the insertion of a gene encoding a fluorescent protein (such as GFP).

In one example, genetic inactivation is achieved by deletion of a portion of the coding region of the OSG-1 gene. For example, some, most (such as at least 50%) or virtually the entire coding region can be deleted. In particular examples, about 5% to about 100% of the gene is deleted, such as at least 20% of the gene, at least 40% of the gene, at least 75% of the gene, or at least 90% of the OSG-1 gene.

Deletion mutants can be constructed using any of a number of techniques. In one example, allelic exchange is employed to genetically inactivate OSG-1 in a nematode. In another technique, the cre-lox system is used for site specific recombination of DNA. In another method, an OSG-1 gene sequence in the nematode genome is replaced with a marker gene, such as green fluorescent protein.

Alternatively, antisense technology can be used to reduce or eliminate the activity of OSG-1. For example, a nematode can be engineered to contain a cDNA that encodes an antisense molecule that prevents OSG-1 from being translated. The term “antisense molecule” encompasses any nucleic acid molecule or nucleic acid analog (e.g., peptide nucleic acids) that contains a sequence that corresponds to the coding strand of an endogenous OSG-1 gene. An antisense molecule also can have flanking sequences (e.g., regulatory sequences). Thus, antisense molecules can be ribozymes or antisense oligonucleotides. A ribozyme can have any general structure including, without limitation, hairpin, hammerhead, or axehead structures, provided the molecule cleaves RNA. Further, gene silencing can be used to reduce the activity of OSG-1.

In one example, a TALEN or CRISPR/Cas9 system is used to inactivate OSG-1.

B. Measuring Gene Inactivation

A nematode having an inactivated OSG-1 gene (referred to herein as ΔOSG-1 or OSG-1 knock out (KO)) can be identified using any method known in the art. For example, PCR and nucleic acid hybridization techniques, can be used to confirm that a nematode has an inactivated OSG-1 gene. Alternatively, real-time reverse transcription PCR (qRT-PCR) can be used for detection and quantification of targeted messenger RNA, such as mRNA of OSG-1 gene in the parent and mutant strains as grown at the same culture conditions. Immunohisto-chemical and biochemical techniques can also be used to determine if a nematode expresses OSG-1 by detecting the expression of the OSG-1 protein encoded by OSG-1. For example, an antibody having specificity for the OSG-1 protein can be used to determine whether or not a particular nematode contains a functional nucleic acid encoding OSG-1 protein. Further, biochemical techniques can be used to determine if a nematode contains an OSG-1 gene inactivation. For example, thermotolerance in response to heat shock conditions can be measured using the methods described herein.

### ARHGEF10

Also provided are recombinant nematodes that expresses functional mammalian ARHGEF10 protein and expresses a fluorescent protein. In some examples, the nematode expresses functional mammalian ARHGEF10 protein in a ΔOSG-1 background; that is, the native OSG-1 is inactivated, but the nematode expresses exogenous ARHGEF10.

The functional mammalian ARHGEF10 expressed by the transgenic nematode can be a functional human ARHGEF10 protein, such as one having at least 90%, at least 91%, at least 92%, at least 93%, at least 94%, at least 95%, at least 96%, at least 97%, at least 98%, at least 99%, or 100% sequence identity to SEQ ID NO: 2. In some examples, the functional human ARHGEF10 protein is encoded by a nucleic acid molecule having at least 90%, at least 91%, at least 92%, at least 93%, at least 94%, at least 95%, at least 96%, at least 97%, at least 98%, at least 99%, or 100% sequence identity to the nucleic acid sequence shown in nucleotides 174-4094 of SEQ ID NO: 1, or to SEQ ID NO: 1.

The promoter used to drive expression of the mammalian ARHGEF10 protein can be native promoter, or a heterologous promoter, such as P_{OSG}1 from C. elegans, or a myo-2 promoter (e.g., see SEQ ID NOS: 5 and 6). In some examples, the promoter has at least 90%, at least 91%, at least 92%, at least 93%, at least 94%, at least 95%, at least 96%, at least 97%, at least 98%, at least 99%, or 100% sequence identity to P_{OSG-1} from C. elegans (SEQ ID NO: 5) or to a myo-2 promoter (e.g., nt 1-1374 of SEQ ID NO: 6).

The transgenic nematodes (ΔOSG-1 or mammalian ARHGEF10 in a ΔOSG-1 background) are engineered to express a fluorescent protein, such as GFP, BFP, CFP, EGFP, r-phycoerythrin, dsRed, mCherry, or YFP

### Variant Sequences

Examples of ARHGEF10 nucleic acid and protein sequences are shown in SEQ ID NO: 1 and 2, and examples of OSG-1 nucleic acid and protein sequences are shown in SEQ ID NO: 3 and 4. In additional exemplary promoter sequences are provided in SEQ ID NOS: 5 and 6. However, the disclosure also encompasses variants of SEQ ID NOS: 1 and 3 which retain the ability to encode an ARHGEF10 or OSG-1 protein, respectively. One skilled in the art will understand that variant OSG-1 nucleic acid sequences can be inactivated in a nematode, and that variant ARHGEF10 sequences can be expressed in a nematode. Variant sequences may contain a single insertion, a single deletion, a single substitution, multiple insertions, multiple deletions, multiple substitutions, or any combination thereof (e.g., single deletion together with multiple insertions). In addition, the degeneracy of the code permits multiple nucleic acid sequences to encode the same protein.

Thus, in some examples, an ARHGEF10 or OSG-1 nucleic acid molecule has at least 80%, at least 85%, at least 90%, at least 95%, at least 97%, at least 98%, or at least 99% sequence identity to any known ARHGEF10 or OSG-1 nucleic acid sequence, such as SEQ ID NO: 1 or 3 or nucleotides 174-4094 of SEQ ID NO: 1. Similarly, in some examples, a promoter nucleic acid molecule has at least 80%, at least 85%, at least 90%, at least 95%, at least 97%, at least 98%, or at least 99% sequence identity to any known promoter nucleic acid sequence, such as SEQ ID NO: 5 or nt 1-1374 of SEQ ID NO: 6.

Examples of ARHGEF10 and OSG-1 protein sequences shown in SEQ ID NOS: 2 and 4, respectively. However, the disclosure also encompasses variants SEQ ID NOS: 2 and 4. One skilled in the art will understand that variant OSG-1 protein sequences can be inactivated, and that variant ARHGEF10 proteins can be expressed in a nematode. Variant sequences may contain a single insertion, a single deletion, a single substitution, multiple insertions, multiple deletions, multiple substitutions, or any combination thereof (e.g., single deletion together with multiple insertions). Such polypeptides share at least 80%, at least 85%, at least 90%, at least 95%, at least 97%, at least 98%, or at least 99% sequence identity to an ARHGEF10 or OSG-1 sequence, such as to SEQ ID NO: 2 or 4, respectively.

In some examples, an OSG-1 sequence that is to be genetically inactivated or the ARHGEF10 to be expressed includes one or more conservative amino acid substitutions. A conservative amino acid substitution is a substitution of one amino acid (such as one found in a native sequence) for another amino acid having similar biochemical properties. Typically, conservative substitutions have little to no impact on the activity of a resulting peptide. In one example, an OSG-1 or ARHGEF10 sequence (such as SEQ ID NO: 2 or 4) includes one or more amino acid substitutions (for example at 1, 2, 5 or 10 residues). Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

### Screening Methods

Provided herein are methods for identifying an agent that increases survival to HS. Such methods can be used to screen more than one test compound simultaneously or contemporaneously, and allows for mixtures of test agents to be tested. In some examples, at least 20, at least 50, at least 96, at least 100, at least 200, at least 300, at least 500, at least 1000, at least 5000, or at least 10,000 compounds are screened simultaneously or contemporaneously. In some examples, the method is performed in a multi-well plate, such as a 6, 12, 48, 96, or 384 well plate(s). In some example, each well of a multi-well plate includes at least 5 worms, such as at least 10, at least 12, at least 15, at least 20, or at least 25 worms/well, such as 5, 6, 7, 8, 9, 10, 11, 12, 13, 14, 15, 16, 17, 18, 19, 20, 21, 22, 23, 24, or 25 worms/well. For example, in a 12-well plate, about 25 worms can be present per well (such as 20-30 worms per well), in a 48-well plate, about 25 worms can be present per well (such as 20-30 worms per well), for a 96-well plate about 10 to 20 worms can be present per well (such as 12 to 15 worms per well), and for a 384-well plate about 5 to 10 worms can be present per well (such as 6 to 9 worms per well). In some examples, the method is automated.

The nematode can be any nematode, such as one of the Caenorhabditis genus, such as C. elegans, C. briggsae, or C. vulgaris. In a specific example, the first and second nematode are C. elegans.

The method can include incubating one or more test agents with a transgenic nematode expressing a fluorescent protein (e.g., GFP in the pharynx) under the myo-2 promoter (or other promoter) referred to herein as the Pmyo-2:GFP nematode for simplicity. The method can also include incubating such a nematode in the absence of the test agent (e.g., as a control).

The nematodes incubated in a culture medium, such as a liquid culture medium (e.g., S buffer), or a solid culture medium (e.g., nematode growth medium (NGM)). In some examples, the culture medium includes E. coli OP50 (e.g., as a food source). E. coli OP50 is a uracil auxotroph.

In some examples, the nematodes are age-synchronized. The fluorescent protein can be the same or different fluorescent proteins. The fluorescent protein expressed by the nematodes can be any fluorescent protein capable of being expressed by a nematode, such as GFP, YFP, or mCherry.

In some examples, the nematodes are incubated in the culture/growth medium for at least 1 day, at least 2 days, at least 3 days, or at least 4 days, such as 2 to 4 days, 3 days, or 4 days. In some examples, the nematodes are incubated in the culture/growth medium at a normal growth temperature for the nematode, such as 15° C. to 25° C., 15° C. to 22° C., or 18° C. to 22° C., such as 20° C.

The survival of the nematodes is measured or determined prior to subjecting them to heat shock conditions, such as at least 12 hours before, at least 18 hours before, or at least 24 hours before. For example, fluorescence from the nematodes can be measured, live and dead nematodes counted (live=fluorescent, dead=nonfluorescent), or combinations thereof. In some examples, digital fluorescence microscopy is used to make such measurements.

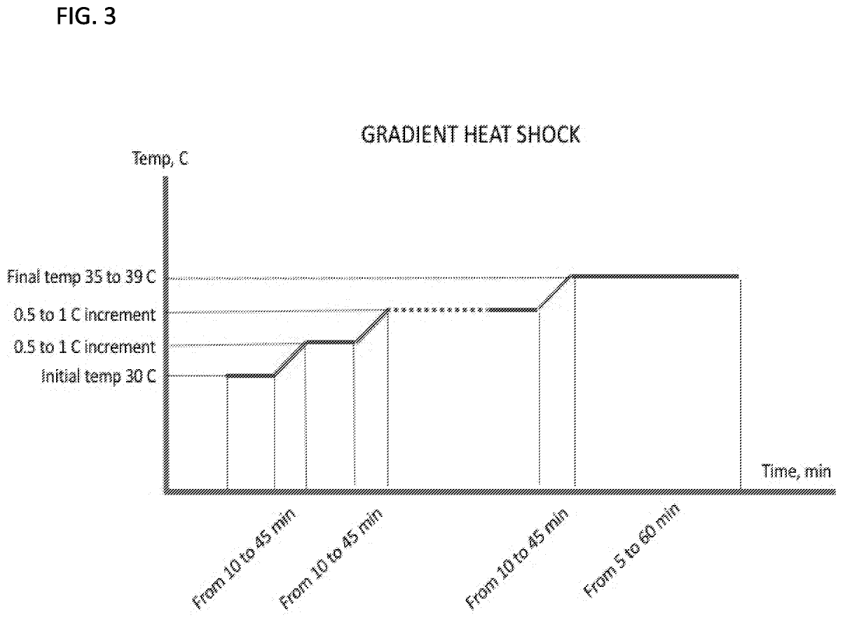

The nematodes are then exposed to HS conditions, such as very weak; weak; medium; strong; or very strong HS conditions (e.g., see Table 1). In some examples, the HS conditions include incubation of the nematodes at a temperature that is at least 10° C. higher than the normal growth temperature for the nematode, such as at least 12° C., at least 15° C., at least 17° C., at least 19° C., or at least 20° C. higher, such as 10° C., 11° C., 12° C., 13° C., 14° C., 15° C., 16° C., 17° C., 18° C., 19° C., or 20° C. higher. In some examples, heat shocking the nematodes includes incubation at a temperature of at least 35° C., such as at least 37° C., or at least 39° C., such as 35-39° C., such as 37° C. (such as 35° C., 36° C., 37° C., 38° C., 39° C., or 40° C.). In some examples, gradient heat shock is used (see FIG. 3 and description herein). In some examples, the nematodes are exposed to heat shock conditions for at least 1 hour, at least 2 hours, at least 3, hours, at least 4 hours, at least 5 hours, or at least 6 hours (such as 1, 1.5, 2, 2.5, 3, 3.5, 4, 4.5, 5, 5.5, or 6 hours), such as about 2 hours for liquid media and 4 hours for solid growth media.

Subsequent to the HS, such as at least 12 hours after, at least 18 hours after, or at least 24 hours after, the survival of the nematodes is measured or determined. For example, fluorescence from the nematodes can be measured, live and dead nematodes counted (live=fluorescent, dead=nonfluorescent), or combinations thereof. In some examples, digital fluorescence microscopy is used to make such measurements.

Test agents that alter this survival ratio:

survival rate of nematodes in the presence of test agent

survival rate of nematodes in the absence of test agent
wherein survival rate=

survival (e.g., # of live) nematodes in the presence of test agent after HS

survival (e.g., # of live) nematodes in the presence of test agent before HS

survival (e.g., # of live) nematodes in the absence of test agent after HS

survival (e.g., # of live) nematodes in the absence of test agent before HS
are agents that can be selected for further analysis.

Provided herein are methods for identifying an agent that targets the actin cytoskeleton. Such methods can be used to screen more than one test compound simultaneously or contemporaneously, and allows for mixtures of test agents to be tested. In some examples, at least 20, at least 50, at least 96, at least 100, at least 500, at least 1000, at least 5000, or at least 10,000 compounds are screened simultaneously or contemporaneously. In some examples, the method is performed in a multiwell plate, such as a 6, 12, 48, 96, or 384 well plate(s). In some example, each well of a multOwell plate includes at least 5 worms, such as at least 10, at least 12, at least 15, at least 20, or at least 25 worms/well, such as 5, 6, 7, 8, 9, 10, 11, 12, 13, 14, 15, 16, 17, 18, 19, 20, 21, 22, 23, 24, or 25 worms/well. For example, in a 12-well plate, about 25 worms can be present per well (such as 20-30 worms per well), in a 48-well plate, about 25 worms can be present per well (such as 20-30 worms per well), for a 96-well plate about 16 worms can be present per well (such as 10-20 worms per well), and for a 384-well plate about 10 worms can be present per well (such as 5-15 worms per well). In some examples, the method is automated.

The nematode can be any nematode, such as one of the Caenorhabditis genus, such as C. elegans, C. briggsae, or C. vulgaris. In a specific example, the first and second nematode are C. elegans.

The method can include incubating one or more test agents with a transgenic first nematode that is functionally deleted (genetically inactivated) for OSG-1 (ΔOSG-1) and expresses a fluorescent protein, and incubating one or more test agents with a transgenic second nematode that expresses a functional mammalian ARHGEF10 protein and a fluorescent protein (wherein the second nematode is in a ΔOSG-1 background). In some examples, the transgenic first nematode that is functionally deleted for OSG-1 and expresses a fluorescent protein, is referred to herein as the OSG-1 KO or the ΔOSG-1 nematode for simplicity. In some examples, the second transgenic nematode that expresses a functional mammalian ARHGEF10 protein and a fluorescent protein in a ΔOSG-1 background, is referred to herein as the ARHGEF10 nematode for simplicity.

The first and the second nematode are incubated separately in a culture medium, such as a liquid culture medium (e.g., S buffer), or a solid culture medium (e.g., nematode growth medium (NGM)). In some examples, the culture medium includes E. coli OP50 (e.g., as a food source). E. coli OP50 is a uracil auxotroph. In some examples, the first and the second nematode are age-synchronized. The fluorescent protein expressed by the first and second nematode can be the same or different fluorescent proteins. The fluorescent protein expressed by the nematodes can be any fluorescent protein capable of being expressed by a nematode, such as GFP, YFP, or mCherry.

In some examples, the nematodes are incubated in the culture/growth medium for at least 1 day, at least 2 days, at least 3 days, or at least 4 days, such as 2 to 4 days, 3 days, or 4 days. In some examples, the nematodes are incubated in the culture/growth medium at a normal growth temperature for the nematode, such as 15° C. to 25° C., 15° C. to 22° C., or 18° C. to 22° C., such as 20° C.

The survival of the first and the second nematode is measured or determined prior to subjecting them to heat shock conditions, such as at least 12 hours before, at least 18 hours before, or at least 24 hours before. For example, fluorescence from the nematodes can be measured, live and dead nematodes counted (live=fluorescent, dead=nonfluorescent), or combinations thereof. In some examples, digital fluorescence microscopy is used to make such measurements.

The first and the second nematode are then exposed to HS conditions, such as very weak; weak; medium; strong; or very strong HS conditions. In some examples, the HS conditions include incubation of the first and second nematodes at a temperature that is at least 10° C. higher than the normal growth temperature for the nematode, such as at least 12° C., at least 15° C., at least 17° C., at least 19° C., or at least 20° C. higher, such as 10° C., 11° C., 12° C., 13° C., 14° C., 15° C., 16° C., 17° C., 18° C., 19° C., or 20° C. higher. In some examples, heat shocking the nematodes includes incubation at a temperature of at least 35° C., such as at least 37° C., or at least 39° C., such as 35-39° C., such as 37° C. (such as 35° C., 36° C., 37° C., 38° C., 39° C., or 40° C.). In some examples, gradient heat shock is used (see FIG. 3). In some examples, the first and the second nematodes are exposed to heat shock conditions for at least 1 hour, at least 2 hours, at least 3, hours, at least 4 hours, at least 5 hours, or at least 6 hours (such as 1, 1.5, 2, 2.5, 3, 3.5, 4, 4.5, 5, 5.5, or 6 hours), such as about 2 hours for liquid media and 4 hours for solid growth media. Exemplary HS conditions are provided in Table 1 below.

The disclosure also provides gradient HS methods, which can be used in the methods for testing agents for their ability to target (e.g., stabilize) actin. An exemplary procedure is illustrated in FIG. 3. Such methods can include incubating a test organism (such as a transgenic nematode provided herein) initially at a first temperature that is at least 5° C. higher than the normal growth temperature for the nematode, such as at least 6° C., at least 7° C., at least 8° C., at least 9° C., or at least 10° C. higher, such as 5° C., 6° C., 7° C., 8° C., 9° C., or 10° C. higher. In some examples, a gradient HS includes incubation at the first temperature of about 30° C., such as at least 27° C., at least 30° C., or 27° C. to 30° C. The incubation at the first temperature can be for at least 1 minute, at least 5 minutes, at least 10 minutes, at least 30 minutes, or at least 60 minutes (such as 1 minute to 60 minutes, 10 minutes to 30 minutes, or 5 minutes to 60 minutes). The gradient HS includes subsequent incubation at a second, higher temperature (e.g., higher than the first temperature), such as a temperature that is at least 10° C. higher than the normal growth temperature for the nematode, such as at least 12° C., at least 15° C., at least 17° C., at least 19° C., or at least 20° C. higher, such as 10° C., 11° C., 12° C., 13° C., 14° C., 15° C., 16° C., 17° C., 18° C., 19° C., or 20° C. higher. In some examples, gradient HS includes incubation at a second temperature of at least 31° C., at least 35° C., at least 37° C., or at least 39° C., such as 31-39° C. or 35-39° C., such as 37° C. (such as 31° C., 32° C., 33° C., 34° C., 35° C., 36° C., 37° C., 38° C., 39° C., or 40° C. ±0.5 or ±0.1° C.). However, the arrival at the second temperature is achieved in stages, such as in temperature increments of 0.1° C. to 1° C. (such as increments of 0.5° C. to 1° C., such as 0.2° C., 0.5° C., or 1° C. increments), wherein the incubation at each temperature increment is performed for at least 10 minutes, at least 30 minutes, or at least 40 minutes (such as 10 to 45 minutes, 10 to 30 minutes or 10 to 20 minutes, such as 10, 15, 20, 25, 30, 35, 40 , or 45 minutes) . For example, the organism can be incubated at 30.5° C. for 10 to 45 minutes, then 31° C. for 10 to 45 minutes, then 31.5° C. for 10 to 45 minutes, then 32° C. for 10 to 45 minutes, then 32.5° C. for 10 to 45 minutes, then 33° C. for 10 to 45 minutes, then 33.5° C. for 10 to 45 minutes, then 34° C. for 10 to 45 minutes, then 34.5° C. for 10 to 45 minutes, then 35° C. for 10 to 45 minutes, then 35.5° C. for 10 to 45 minutes, then 36° C. for 10 to 45 minutes, then 36.5° C. for 10 to 45 minutes, then 37° C. for 10 to 45 minutes, then 37.5° C. for 10 to 45 minutes, then 38° C. for 10 to 45 minutes, and then 38.5° C. for 10 to 45 minutes. Once the second temperature is achieved, the test organism (e.g., transgenic nematode) is then maintained at the second temperature of 31° C. to 39° C. (such as 31° C., 32° C., 33° C., 34° C., 35° C., 36° C., 37° C., 38° C., or 39° C. ±0.5 or ±0.1° C.) for at least 5 minutes, at least 10 minutes, at least 30 minutes, at least 60 minutes, or at least 90 minutes (such as 5 to 30 minutes 5 to 60 minutes, 5 to 120 minutes, 10 to 30 minutes, or 10 to 60 minutes). Subsequent to the HS, such as at least 12 hours after, at least 18 hours after, or at least 24 hours after, the survival of the first and the second nematode is measured or determined. For example, fluorescence from the nematodes can be measured, live and dead nematodes counted (live =fluorescent, dead=nonfluorescent), or combinations thereof. In some examples, digital fluorescence microscopy is used to make such measurements.

Test agents that alter this survival ratio:

survival rate of second nematode (ARHGEF10) in the presence of test agent

survival rate of first nematode (ΔOSG-1) in the presence of test agent

as compared to this ratio

survival rate of second nematode (ARHGEF10) in the absence of test agent

survival rate of first nematode (ΔOSG-1) in the absence of test agent

are agents that target actin. For example, a ratio of about 2.7 has been calculated for:

survival rate of second nematode (ARHGEF10) in the absence of test agent

survival rate of first nematode (ΔOSG-1) in the absence of test agent

That is, ARHGEF10 worms have a higher survival rate % (48±3% alive worms) than ΔOSG-1 worms (18±5% alive worms), thus 48/18=2.7 ratio. Thus, if the survival ratio in the presence of the test agent is greater or lower than about 2.7 (such as at least 3, at least 3.5, or at least 4, or less than 2.5, less than 2 or less than 1.5), this indicates the agent is one that can target the actin cytoskeleton. In some examples, such agents are selected for further examination. If the ratio remains at about 2.7 (e.g., survival rate for both sets of worms increase or decreases), this indicates the agent is one that acts independently of actin. Thus, in some examples, if the ratio remains at about 2.7 (e.g., ±0.3), this indicates the agent is one that acts independently of actin.

It has been observed that ΔOSG-1 worms have lower survival rates (18±5% alive worms) than ARHGEF10 worms (48±3% alive worms). Therefore compounds that alter the 48/18=2.7 ratio are considered potential hits for agents that affect the actin cytoskeleton. Experiments can be repeated at least in triplicate to achieve statistical significance.

### Kits

The present disclosure provides kits, such as kits that include the recombinant or transgenic nematodes provided herein. Such nematodes can be in separate containers, for example in a medium for culturing, storing, or growing the nematode.

In one example, the kit includes (1) a transgenic first nematode that is functionally deleted for OSG-1 (e.g., does not express functional OSG-1) and expresses a fluorescent protein and (2) a transgenic second nematode that expresses functional mammalian ARHGEF10 protein and expresses a fluorescent protein (which can be in a ΔOSG-1 background). In some examples the first and second nematode are C. elegans, C. briggsae, or C. vulgaris. In a specific example, the first and second nematode are C. elegans. In some examples, the fluorescent protein expressed by the first and second transgenic nematodes is GFP, BFP, CFP, EGFP, r-phycoerythrin, dsRed, mCherry, or YFP. In some examples, the first and second transgenic nematodes express the same fluorescent protein, and in other examples, first and second transgenic nematodes express different fluorescent proteins. In some examples, the first and second transgenic nematodes are in separate containers.

Such kits can include additional reagents, such as one or more multiwell plates (such as a 6, 12, 48, 96, or 384 well plate(s)), culture medium suitable for growth or storage of the transgenic nematodes (such as a liquid or solid medium), or combinations thereof. In some examples, the kit includes E. coli OP50 or another food source for the nematode. In some example, the kit includes positive and negative controls for the assay, such as a positive control known to target the actin cytoskeleton, and/or a negative control known to not target the actin cytoskeleton. In some examples, the positive control alters the ratio of survival of the transgenic nematode expressing functional mammalian ARHGEF10 protein to survival of the ΔOSG-1 transgenic nematode f, as compared to such a ratio without the positive control. In some examples, the negative control does not alter the ratio of survival of the transgenic nematode expressing functional mammalian ARHGEF10 protein to survival of the ΔOSG-1 transgenic nematode, as compared to such a ratio without the negative control.

### EXAMPLE 1

### Generation of Recombinant C. elegans with OSG-1 Gene Knock Out

This Example describes the methods used to functionally delete the OSG-1 gene and to express GFP in C. elegans. The methods are also provided in Duan and Sesti (2015) Genetics 199, 487-496.

The RB1560 strain (ok1894 allele or OSG-1 knock out) was obtained from the The Caenorhabditis Genetics Center (CGC).

GFP was expressed from the promoter of the OSG-1 gene from C. elegans (P_{OSG-1}) or from the myo-2 promoter.

POSG-1 is a ˜2.3 kb genomic fragment upstream the OSG-1 gene in C. elegans containing the open reading frame. This promoter was amplified by PCR with forward primer AACTGCAGTGTGACTTGGGATCATCGGA (SEQ ID NO: 7) and reverse primer CGGGATCCTTGAGCCACAGGGGAGCCAATG from 5′ to 3′ (SEQ ID NO: 8). To generate P_{OSG-1}:GFP, P_{OSG}-1 was subcloned in pPD95.75 Fire vector using Pstl and BamHI restriction sites.

The myo-2:GFP cDNA (pPD118.33) was from Addgene.

To generate the OSG-1 KO/GFP transgenic worm (ok1894; P_{OSG-}1:GFP; rol-6) (formal name: FDX 2533 sesEx2533) RB1560 worms were injected with P_{OSG}-1: GFP; and rot-6 transformation marker.

To generate the OSG-1 KO/myo:GFP transgenic worm (ok1894; myo-2:GFP), RB1560 worms were injected with myo-2:GFP.

Worms were grown in NGM in the presence of OP50 E. coli bacteria. Mutants expressing GFP were identified by screening for the rol-6 phenotype marker or for GFP in the pharynx using fluorescence microscopy.

### EXAMPLE 2

### Generation of Recombinant C. elegans Expressing Human ARHGEF10

This Example describes the methods used to express human ARHGEF10 cDNA and GFP in C. elegans.

The RB1560 strain, (ok1894 allele or OSG-1 knock out) was obtained from The Caenorhabditis Genetics Center (CGC).

Human ARHGEF10 cDNA (e.g., SEQ ID NO: 1) was from Source BioScience LifeSciences. Human ARHGEF10 cDNA was expressed from the promoter of the OSG-1 gene from C. elegans (PosG-1).

GFP was expressed from the myo-2 or the P_{OSG-1} promoter as described in Example 1.

To generate the P_{OSG-1}:ARHGEF10 transgenic worm, human ARHGEF10 was subcloned in P_{OSG-1}:GFP using Hind III and Kpn I restriction sites.

To generate the ARHGEF10/GFP transgenic worm (ok1894, P_{OSG-1}:ARHGEF10; P_{OSG-}1: GFP; rot-6) RB1560 worms were injected with P_{OSG-1}:ARHGEF10; P_{OSG-1}:GFP; and rot-6 transformation marker.

To generate the ARHGEF10/myo:GFP transgenic worm (ok1894, P_{OSG-1}:ARHGEF10; myo-2:GFP), RB1560 worms were injected with P_{OSG-1}:ARHGEF10 and myo-2:GFP.

Worms were grown in NGM in the presence of OP50 E. coli bacteria. Mutants expressing GFP were identified by screening for the rol-6 phenotype or for GFP in the pharynx using fluorescence microscopy. The presence of ARHGEF10 was confirmed by PCR.

### EXAMPLE 3

### Screening Method with Solid Agar Media

This Example describes methods that can be used to test agents in the presence of the transgenic C. elegans generated in Examples 1 and 2. Here, a solid media (such as nematode growth medium, NGM) is used. In some examples, the worms expressing the genes from the P_{OSG-1} promoter are used. In some examples, the worms expressing the genes from the myo-2 promoter are used (ARHGEF10/myo-2:GFP and OSG-1 KO/myo-2:GFP). Both promoters are expected to provide similar results.

NGM plates were prepared as follows. Mix 3 g NaCl, 17 g agar, and 2.5 g peptone in a 2 liter flask and add 975 ml H2O. Autoclave for 50 min. Cool flask in 55° C. water bath for 15 min. Add 1 ml 1 M CaC12, 1 ml 5 mg/ml cholesterol in ethanol, 1 ml 1 M MgSO4 and 25 ml 1 M KPO4 buffer. Mix well. Using sterile procedures, dispense the NGM solution into plates (such as 6 cm Petri plates 12-well plates, 96-well plates, or 384-well plate, such as 200 μL per well of a 96-well pate, or 90 μL per well of a 384-well plate), for example using a peristaltic pump. In some examples, plates or wells are filled 2/3 full with the NGM. Plates can be left at room temperature for 1 to 3 days before use to allow for detection of contaminants, and to allow excess moisture to evaporate. Plates stored in an air-tight container at room temperature are usable for several weeks. A solution containing the desired test compound can be added to appropriate plates or individual wells and can be allowed to dry. In some examples, compounds are diluted in solvent. Compounds can be directly dissolved in the liquid agar or alternatively added to the plate after agar is solid. The plates are seeded with E. coli OP50. The bacteria are prepared fresh and seeded when they are at the end of the exponential phase and the beginning of the stationary phase.

Culture plates containing solid NGM media and E. coli OP50 are seeded with ARHGEF10/GFP or OSG-1 KO/GFP worms (with the ARHGEF10/GFP and OSG-1 KO/GFP worms in separate wells, e.g., see FIG. 2B). At day 0 plates containing one or more test compounds are prepared. For example, individual test compounds can be tested in individual wells of the plate. Compounds can be directly dissolved in the liquid agar or alternatively added to the plate after agar is solid. Then plates are seeded with freshly grown OP50 E. coli bacteria.

At day 1 age-synchronized ARHGEF10/GFP and OSG-1 KO/GFP worms in liquid M9 media, are seeded (pipetted) onto pre-treated individual wells of NGM 12-well plates at a concentration 25 worms/well (or 12-15 worms/well in a 96-well plate or 6-9 worms/well in a 384-well plate). The concentration of worms in the liquid media can be calculated by pipetting 2μL on a glass slide and counting the worms under a dissection microscope (the final concentration is obtained as an average of 3 measurements). The seeded plates are kept at 20° C.

At day 4, worms in individual wells are imaged under a dissection microscope equipped with a GFP lamp and a digital camera (1 image/well). Note that for each compound there are two images, one with ARHGEF10/GFP worms and one with OSG-1 KO/GFP worms. At the end of this procedure the plates are returned to the incubator and incubated at 20° C. for 24 hours.

At day 5 plates are transferred to a second incubator and maintained at 37° C. for 4 hours (heat shock). They are then returned to the 20° C. incubator and incubated for 24 hours. Alternatively, a heat shock procedure described in Example 5 is used.

At day 6 plates are imaged under the GFP light as done at day 4. For each well, the number of fluorescent worms before (NB) and after (NA) heat shock is counted and survival rate is calculated as: NA/NB.

It has been observed that OSG-1 KO/GFP worms have lower survival rates (18±5% alive worms) than ARHGEF10/GFP worms (48±3% alive worms). Therefore compounds that alter the 48/18=2.7 ratio are considered potential hits for agents that affect the actin cytoskeleton. Such agents can be selected for further experimentation, such as in vivo. Experiments can be repeated at least in triplicate to achieve statistical significance.

### EXAMPLE 4

### Screening Method with Liquid Media

This Example describes methods that can be used to test agents in the presence of the transgenic C. elegans generated in Examples 1 and 2. Here, a liquid media (such as S buffer) is used. In some examples, a liquid media is used, for example for compounds that are poorly soluble or require large amounts. In some examples, the worms expressing the genes from the P_{OSG}-1 promoter are used. In some examples, the worms expressing the genes from the myo-2 promoter are used (ARHGEF10/myo-2:GFP and OSG-1 KO/myo-2:GFP). Both promoters are expected to provide similar results.

M9 Buffer is prepared as follows. To 1 L of DI H2O, add under constant stirring 3g KH2PO4, 6g Na2HPO4, and 5g NaCl, and autoclave and allow to cool. Add 1mL of 1 M MgSO₄.

Liquid media is prepared as follows.
1. S Basal [5.85 g NaCl, 1 g K₂ HPO₄, 6 g KH₂PO₄, 1 ml cholesterol (5 mg/ml in ethanol), H₂O to 1 L. Sterilize by autoclaving].
2. 1 M Potassium citrate pH 6.0 (20 g citric acid monohydrate, 293.5 g tri-potassium citrate monohydrate, H₂O to 11 L. Sterilize by autoclaving.
3. Trace metals solution [1.86 g disodium EDTA, 0.69 g FeSO₄·7 H₂O, 0.2 g MnCl2·4 H₂O, 0.29 g ZnSO₄·7 H₂O, 0.025 g CuSO₄·5 H₂O, H₂O to 1 L. Sterilize by autoclaving. Store in the dark.]
4. 1 M CaCl₂[55.5 g CaCl₂ in 1 L1420. Sterilize by autoclaving.]
5. S Medium [1 L S Basal, 10 ml 1 M potassium citrate pH 6, 10 ml trace metals solution, 3 ml 1 M CaCl₂, 3 ml 1 M MgSO₄. Add components using sterile technique; do not autoclave.]
6. Add concentrated (pelleted) E. coli OP50.
Age synchronization is performed as follows (see Duan and Sesti (2013) J Vis Exp, e50435):
   1. Worms described in Examples 1 and 2 are grown in standard 6 cm NGM dishes+OP50 E. coli until a large population of gravid adults is reached (typically 3-5 days).
   2. Worms are collected in 50 ml tubes, washed in M9 buffer and treated with 10 volumes of basic hypochlorite solution (0.25 M NaOH, 1% hypochlorite freshly mixed).
   3. Worms are incubated at room temperature until fully dissolved (approximately 10 minutes)
   4. The eggs (and carcasses) are collected by centrifugation at 400g for 5 minutes. This procedure is repeated 4 times and then the eggs are incubated overnight in M9 buffer.

Using sterile procedures, dispense the M9 buffer into plates (such as 6 cm Petri plates 12-well plates, 96-well plates, or 384-well plates, such as 200 μL per well of a 96-well pate, or 90 per well of a 384-well plate).

At day 1 age-synchronized ARHGEF10/GFP and OSG-1 KO/GFP worms (or ARHGEF10/myo-2:GFP and OSG-1 KO/myo-2:GFP worms) in liquid M9 media, are seeded (pipetted) onto pre-treated individual wells of 96-well plates containing S buffer, E. coli OP50 pellets and test compound(s) at a concentration 6-12 worms/well (or 6-9 worms/well in a 384-well plate). The concentration of worms in M9 can be calculated by pipetting 2 μL on a glass slide and counting the worms under a dissection microscope (the final concentration is obtained as an average of 3 measurements). The plates containing the worms, test compounds and OP50 bacteria are incubated at 20° C.

At day 4, worms in individual wells are transferred to NGM 96-well plates containing OP50 E. coli and imaged under a dissection microscope equipped with a GFP lamp and a digital camera (1 image/well). Then the plates are incubated at 20° C. for 24 hours.

At day 5 plates are transferred to a second incubator and maintained at 37° C. for 4 hours (heat shock). Alternatively, a heat shock procedure described in Example 5 is used. The plates are then returned to a 20° C. incubator and incubated for 24 hours.

At day 6 plates are imaged under the GFP light as done at day 4. For each well, the number of fluorescent worms before (NB) and after (NA) heat shock is counted and survival rate is calculated as: NA/NB.

In some examples, at day 5, plates are transferred to a water bath incubator and maintained at 37° C. for 2 hours (heat shock). Worms are returned to 20° C. for 2 hours and then worms of each individual well are transferred to individual wells of NGM 96-well plates containing OP50 bacteria and incubated for 24 hours. At day 6 plates are imaged under the GFP light.

In some examples, at day 5, plates are transferred to a water bath incubator and maintained at 37° C. for 2 hours (heat shock). They are then returned to the 20° C. incubator and incubated for 24 hours. At day 6 plates are imaged under the GFP light.

It has been observed that OSG-1 KO/GFP worms have lower survival rates (18±5% alive worms) than ARHGEF10/GFP worms (48±3% alive worms). Therefore compounds that alter the 48/18=2.7 ratio are considered potential hits for agents that affect the actin cytoskeleton. Such agents can be selected for further experimentation, such as in vivo. Experiments can be repeated at least in triplicate to achieve statistical significance.

### EXAMPLE 5

### Screening Method with Solid Agar Media

This Example describes methods that can be used to test agents in the presence of a transgenic C. elegans expressing a fluorescent protein in a WT background, such as C. elegans N2 expressing GFP under myo-2 promoter in the pharinx (herein named pmyo-2:GFP). Here, a solid media (such as nematode growth medium, NGM) is used.

NGM plates were prepared as described in Example 3. Culture plates containing solid NGM media and E. coli OP50 are seeded with Pmyo-2:GFP worms. At day 0 plates containing one or more test compounds are prepared. For example, individual test compounds can be tested in individual wells of the plate. Compounds can be directly dissolved in the liquid agar or alternatively added to the plate after agar is solid. Then plates are seeded with freshly grown OP50 E. coli bacteria. Control plates include no test compounds.

At day 1 age-synchronized Pmyo-2:GFP worms in liquid M9 media, are seeded (pipetted) onto pre-treated individual wells of NGM 12-well plates at a concentration 25 worms/well (or 12-15 worms/well in a 96-well plate or 6-9 worms/well in a 384-well plate). The concentration of worms in the liquid media can be calculated by pipetting 2μL on a glass slide and counting the worms under a dissection microscope (the final concentration is obtained as an average of 3 measurements). The seeded plates are kept at 20° C.

At day 4, worms in individual wells are imaged under a dissection microscope equipped with a GFP lamp and a digital camera (1 image/well). At the end of this procedure the plates are returned to the incubator and incubated at 20° C. for 24 hours.

At day 5 plates are transferred to a second incubator and subject to different heat shock conditions (e.g., as listed in Table 1 or a gradient heat shock as described in Example 7 and shown in FIG. 3). They are then returned to the 20° C. incubator and incubated for 24 hours.

At day 6 plates are imaged under the GFP light as done at day 4. For each well, the number of fluorescent worms in the absence (NB) and presence (NA) of the test compound after heat shock is counted and survival rate is calculated as: NA/Total number of worms in the well with test compound measured prior to HS and NB//Total number of worms in the well without test compound measured prior to HS.

Compounds that improve survival under different HS conditions (very-weak, weak etc.) are considered potential hits for agents that affect the actin cytoskeleton (e.g., stabilize F-actin). Such agents can be selected for further experimentation, such as in vivo. Experiments can be repeated at least in triplicate to achieve statistical significance.

### EXAMPLE 6

### Screening Method with Liquid Media

This Example describes methods that can be used to test agents in the presence of C. elegans expressing a fluorescent protein in a WT background, such as C. elegans N2 expressing GFP under myo-2 promoter in the pharinx. Here, a liquid media (such as S buffer) is used. In some examples, a liquid media is used, for example for compounds that are poorly soluble or require large amounts.

M9 Buffer and liquid media are prepared as described in Example 4. Age synchronization is performed as described in Example 4. Using sterile procedures, dispense the M9 buffer into plates (such as 6 cm Petri plates 12-well plates, 96-well plates, or 384-well plates, such as 200 per well of a 96-well pate, or 90 μL per well of a 384-well plate).

At day 1 age-synchronized Pmyo-2:GPF worms in liquid M9 media, are seeded (pipetted) onto pre-treated individual wells of 96-well plates containing S buffer, E. coli OP50 pellets and test compound(s) at a concentration 6-12 worms/well (or 6-9 worms/well in a 384-well plate). The plates containing the worms, test compounds and OP50 bacteria are incubated at 20° C. Control plates include no test compounds.

At day 4, worms in individual wells are transferred to NGM 96-well plates containing OP50 E. coli and imaged under a dissection microscope equipped with a GFP lamp and a digital camera (1 image/well). Then the plates are incubated at 20° C. for 24 hours.

At day 5 plates are transferred to a second incubator and subjected to various HS conditions (e.g., as listed in Table 1 or a gradient heat shock as described in Example 7 and shown in FIG. 3). The plates are then returned to a 20° C. incubator and incubated for 24 hours.

At day 6 plates are imaged under the GFP light as done at day 4. For each well, the number of fluorescent worms in the absence (NB) and in the presence (NA) of the test compound after heat shock is counted and survival rate is calculated as: NA/Total number of worms in the well with test compound measured prior to HS and NB//Total number of worms in the well without test compound measured prior to HS.

In some examples, at day 5, plates are transferred to a water bath incubator and maintained at 37° C. for 2 hours (heat shock). Worms are returned to 20° C. for 2 hours and then worms of each individual well are transferred to individual wells of NGM 96-well plates containing OP50 bacteria and incubated for 24 hours. At day 6 plates are imaged under the GFP light.

In some examples, at day 5, plates are transferred to a water bath incubator and maintained at 37° C. for 2 hours (heat shock). They are then returned to the 20° C. incubator and incubated for 24 hours. At day 6 plates are imaged under the GFP light.

Compounds that improve survival under different HS conditions (very-weak, weak etc.) are considered potential hits for agents that affect the actin cytoskeleton (e.g., stabilize F-actin). Such agents can be selected for further experimentation, such as in vivo. Experiments can be repeated at least in triplicate to achieve statistical significance.

### EXAMPLE 7

### Heat Shock Protocols

This Example describes heat shock methods that can be used with the methods described in Examples 3-6.

A solid or liquid media can be used, such as NGM or S Medium, as described above. The media includes OP50 E. coli. Worms and test agent(s) are added to the media as described above.

On one example, a fixed heat shock temperature procedure is used. Plates containing worms and vehicle or drug are incubated at 35° C. to 39° C. or incubated at variable length of times according to Table 1. Such a method can be performed in a PCR machine.

TABLE 1
Exemplary heat shock (HS) protocol

| | Temperature in Celsius | | | | |
|---|---|---|---|---|---|
| Time (hours) | 35 | 36 | 37 | 38 | 39 |
| 1 | A | A | A | A | A |
| 1:30 | A | A | A | A | B |
| 2 | A | A | B | B | B |
| 2:30 | A | B | B | B | C |
| 3 | B | B | C | C | D |
| 3:30 | B | C | C | C | D |
| 4 | C | C | C | D | E |
| 4:30 | C | C | C | D | E |
| 5 | C | D | D | E | E |
| 6 | D | D | E | E | E |

Strength of the HS: A very weak; B weak; C medium, D strong; E very strong.

In another example, a gradient heat shock temperature procedure is used. Plates containing worms and vehicle or drug are incubated 30° C. The temperature is raised to 35° C. to 39° C. in 0.5 to 1° C. increments in time increments from 10 minutes to 45 minutes. The temperature is maintained at the final temperature (35° C. to 39° C.) for 5 minutes to 60 minutes (FIG. 3). Such a method can be performed in a PCR machine.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

1. A method for identifying an agent that targets actin, comprising:
incubating in a culture medium a test agent with a transgenic nematode that expresses a fluorescent protein in a wild-type background;
determining survival of the transgenic nematode prior to heat shock conditions;
exposing the transgenic nematode to heat shock conditions; and
determining survival rate for the transgenic nematode following the heat shock conditions, wherein a test agent that alters the ratio of survival of the transgenic nematode in the test agent after the heat shock conditions to survival of the transgenic nematode in the test agent before the heat shock conditions is an agent that targets actin.

2. A method for identifying an agent that targets actin, comprising:
incubating a test agent with a transgenic first nematode that is functionally deleted for OSG-1 and expresses a fluorescent protein;
incubating the test agent with a transgenic second nematode that expresses a functional mammalian ARHGEF10 protein and the fluorescent protein but does not express functional OSG-1;
wherein the first and the second nematode are incubated separately in a culture medium,
determining survival of the first and the second nematode prior to heat shock conditions;
exposing the first and the second nematode to heat shock conditions; and
determining survival rate for the first and the second nematode following the heat shock conditions, wherein a test agent that alters the ratio of survival of the transgenic second nematode in the test agent to survival of the transgenic first nematode in the test agent as compared to the ratio in an absence of the test agent is an agent that targets actin.

3. The method of claim 1, wherein the culture medium is a liquid culture medium.

4. (canceled)

5. The method of claim 1, wherein the culture medium is a solid culture medium.

6.-8. (canceled)

9. The method of claim 2, wherein a native OSG-1 sequence that is functionally deleted comprises at least 95% sequence identity to SEQ ID NO: 3.

10. The method of claim 2, wherein the functional mammalian ARHGEF10 protein comprises a functional human ARHGEF10 protein.

11. (canceled)

12. (canceled)

13. The method of claim 1, wherein the transgenic nematode is C. elegans, C. briggsae, or C. vulgaris.

14. The method of claim 1, wherein the heat shock conditions comprise incubation of the transgenic nematode at a temperature of 35° C. to 39° C. for at least 1 hour.

15. The method of claim 1, wherein determining survival rate comprises measuring fluorescence generated from the fluorescent protein from the transgenic nematode.

16. The method of claim 1, wherein determining survival rate for the transgenic nematode prior to the heat shock conditions is performed at least 12 hours before exposing the transgenic nematode to the heat shock conditions.

17. The method of claim 1, wherein determining survival for the transgenic nematode following the heat shock conditions is performed at least 12 hours after exposing the transgenic nematode or the first and the second nematode to the heat shock conditions.

18. The method of claim 2, wherein the transgenic second nematode that expresses a functional mammalian ARHGEF10 protein and the fluorescent protein is further functionally deleted for OSG-1.

19. An isolated transgenic nematode that expresses a functional mammalian ARHGEF10 protein and a fluorescent protein but does not express functional OSG-1.

20. The isolated transgenic nematode of claim 19, wherein the functional mammalian ARHGEF10 protein comprises at least 95% sequence identity to SEQ ID NO: 2.

21. The isolated transgenic nematode of claim 19, wherein the functional mammalian ARHGEF10 protein is encoded by a nucleic acid molecule comprising at least 95% sequence identity to nucleotides 174-4094 of SEQ ID NO: 1.

22. The isolated transgenic nematode claim 19, wherein the nematode is further functionally deleted for OSG-1.

23. A kit comprising:
a transgenic first nematode that is functionally deleted for OSG-1 and expresses a fluorescent protein; and
the isolated transgenic nematode of claim 19.

24. The kit of claim 23, further comprising:
a multiwell plate;
a culture medium;
a positive control;
a negative control;
E. coli; or
combinations thereof.

25. The method of claim 1, wherein exposing the transgenic nematode to heat shock conditions comprises:
incubating a test eukaryotic organism at a temperature of ° about 30° C. for 1 minute to 60 minutes;
increasing the temperature to a temperature of 31° C. to 39° C. in temperature increments of 0.1° C. to 1° C., wherein each temperature increment is performed for 1 minute to 45 minutes; and
maintaining the temperature of 31° C. to 39° C. for 5 minutes to 120 minutes.

26. The heat shock method of claim 25, wherein the method comprises:
incubating the test eukaryotic organism at a temperature of about 30° C. for 1 minute to 60 minutes;
increasing the temperature to a temperature of 35° C. to 39° C. in temperature increments of 0.5° C. to 1° C., wherein each temperature increment is performed for 10 to 45 minutes; and
maintaining the temperature of 35° C. to 39° C. for 5 minutes to 60 minutes.


SEQUENCE LISTING (8 sequences)

SEQ ID NO: 1 - DNA, 5508 bp, Homo sapiens
  CDS (174)..(4094)
gacgcgggggacgcgggggacggcggggaacggcgggggacggcggggaacagcgggggacggcggggaacagcgggggacggcggggaacagcaggggactgcgggtcgcgtcctggccgccgaggagctccttcccttaacagagctgagagaggcatctggagctgcagcatggaccagcgagagcccctgcctcccgctcctgcagaaaatgaaatgaaatatgataccaataataatgaagaggaagagggagaacagttcgattttgacagtggagatgaaatcccagaagcggacagacaggccccctccgcccctgagacaggaggtgctggagccagtgaagcccctgcacccacaggaggtgaggatggagctggagcagaaaccaccccagtggcagagcctactaagctggtgctcccgatgaaagtcaacccatattctgtcatcgacatcacgccattccaggaggaccagccgcccacccccgtgcccagcgctgaggaggagaatgtgggtctccatgtgccctgcgggtacttggtgcctgtaccctgcggctatgcggtgccctccaacctgcccctcctgctgcccgcctactccagcccggtcatcatctgcgccacgtccctggacgaagaagcagaaacaccagaagtcacagaagatcgccagcccaattctctgagttccgaggagcctccaaccagtgaagatcaagtcggtcgagaggacagcgcacttgcccgctgggccgcagacccggccaacacagcctggatggagaatccagaggaagcaatttacgatgacgttccaagggaaaactcagactctgaaccagatgaaatgatttatgatgatgttgagaatggggatgaaggtggaaacagctccttggaatacggatggagttcgagtgaatttgaaagttacgaagagcagagtgactcggagtgcaagaatgggattcccaggtccttcctgcgcagcaaccacaaaaagcaaatgcagaagctcgtgaaggccgcgaaggacggcaccaaggacgggctggagaggaccagggcagccgtgaagaggggccgctccttcatcaggaccaagtctctcatcgcacaggatcacagatcttctcttgaggaagaacagaatttcttcattgatgttgactgcaagcacccggaagccatcttgaccccgatgcccgagggtttatctcagcagcaggttgtaagaagatatatactgggttcagttgtcgacagtgaaaagaactacgtagatgctcttaagaggattttggagcaatatgagaagccgctgtctgagatggagccaaaggttctgagtgagaggaagctgaagacggtgttctaccgagtcaaagagatcctgcagtgccactcgctatttcagatcgcgctggccagccgcgtttccgagtgggactccgtggaaatgataggcgatgtcttcgtggcttcgttttctaagtccatggtgctggatgcatacagtgaatatgtgaacaatttcagcacagccgtggcagtcctcaagaaaacatgtgccacaaagcccgcttttcttgaatttttaaagcaggaacaggaggccagccccgatcgaaccacgctctacagcctgatgatgaagcccatccagaggttcccacagttcatcctcctgcttcaggacatgctgaagaacacctccaaaggccaccccgacaggctgcctcttcagatggccctgacagagctcgaaacactagcagagaagttaaatgaaagaaagagagatgctgatcaacgctgtgaagtgaagcaaatagccaaagccataaacgaaagatacctgaacaagcttctcagcagtggaagccgatacctcattcgatcagatgatatgatagaaacagtttacaacgacagaggagagattgttaaaaccaaagaacgccgagtcttcatgttaaatgatgtgttaatgtgtgccaccgtcagctcacgcccctctcatgacagccgtgtgatgagcagccagaggtacttgctgaagtggagcgttccactgggacatgtggacgccatcgagtatggcagcagcgcaggcacgggcgagcacagcaggcaccttgccgttcacccgccggagagcctggccgtggttgctaacgcgaaaccaaacaaagtttacatggggccaggacaactgtatcaagatttacaaaacttgttgcatgacttaaatgtaattggccaaatcactcagctgataggaaaccttaaaggaaactatcagaacttaaaccagtcagtagcccatgactggacatcaggtttacaaaggcttattttgaagaaagaagatgaaatcagagctgcggactgctgcagaattcagttacagcttcccgggaagcaggacaaatctgggcgaccgacgttctttacagctgtgttcaatacgttcacccctgccatcaaggagtcctgggtcaacagcttacagatggccaagctcgccctagaagaggagaaccacatgggctggttctgtgtggaagacgatgggaatcacattaaaaaggagaagcatcctctcctcgtcggacacatgcccgtgatggtggccaagcagcaggagttcaagattgaatgtgctgcttataaccctgaaccttacctaaataatgaaagccagccagattcattttccacggcacatggtttcctgtggatcggaagttgcacccatcaaatgggtcagattgccatcgtctcgtttcaaaattccactcccaaagtcattgagtgcttcaacgtggaatctcgcatcctgtgcatgctgtacgttcccgtcgaggagaagcgcagagagcctggggcacccccggaccccgagaccccggccgtgagagcttctgatgtccccacgatctgtgtagggacggaggagggaagcatttccatttataaaagcagtcaaggctccaagaaagtgagacttcagcactttttcactcctgagaagtccacagtcatgagcctggcttgcacgtctcagagcctgtacgctggcctggtcaacggggcagtcgccagctacgccagagccccagatggatcctgggattcagaacctcaaaaagtgatcaagttaggcgtcctaccagttagaagtctactcatgatggaagacacgttgtgggcggcttccggaggtcaagtcttcatcatcagtgtggagactcatgctgtagagggtcagctggaggcccaccaggaggaaggcatggtgatctcccacatggccgtgtccggcgtcgggatctggattgccttcacctcagggtccacgctccgcctttttcacacggaaactctcaagcacctgcaggacatcaacatcgccacccctgttcacaacatgctgccagggcaccagcggctgtcggtgacgagcctgctcgtctgccacggattgctgatggtcggcaccagcctgggagtcctcgtggccctgccggtcccacgtctgcaagggattcccaaagtgaccggaagaggcatggtctcctaccatgcacacaacagtcctgtcaaattcatcgtcctggccacggctctgcacgagaaagacaaggacaaatccagggacagcctggctcctggccccgagcctcaggacgaagaccagaaggacgcacttccgagtggaggagctggttcatctctgagccagggtgaccctgacgcagccatctggttgggagattcgctgggatcgatgactcagaaaagcgacctgtcctcctcatctgggtccctgagcttgtctcacggctccagctctctagagcacagatcagaggacagcaccatctatgatctcctgaaggatcctgtctcgctgagaagcaaagcacgccgggccaagaaagccaaggccagctcggcgctggtggtctgtggagggcagggccaccgccgggtgcacaggaaggcccggcagccccaccaggaagagctggcgccgaccgtcatggtctggcagatccctctgctgaatatataagcaggacggccgccttctgctgtcagaatttgcaatcaagggtgacttctcagctaatcctacagcctgagtggttaagctgtgtctacactggttgggaataaattaaaaacagtatttgggggagaaacgtgcaatagcgtaatggtggtgtccctgccaattccttccttctcttctgtacagcagaagtaattacaagcacttctcacgaaggcagaagactgatgcaattttcgagtaattgagtgcagttctgggaaaataccacattctttttgactgctgtagtccatatatgaatactaaatgttaaacttcatcagcgtcagacctattgtatcatattagagaatttgcagactaagaatttatgagaaaatatatgtattcagtagtgcaggcatttattaacaattcttaaaagttttacctgattcagattcacgacttttatttatattctatatttttgaatttcagagtaaaatttgttaacaattttaaaagccaggtaacacctaccagtccagttagcatgatttgctttcagaagtgagctgggttttccaaagtggtataatgtgtgtactgtatattttaacaaagtaatatttttgtattgcatttttctattaaaaaattaacagttaatgtttcagtcaatgtattatctgtagcatttcacaaataatgtttgctttgaaccaaaatgctcagtgcctatcaacatttggactcaagcatcaacaccaaattattcctcccttctcgtataaatagagtgactatccacaggagaaaagtgtgtgctttagtattagaggagataggcagagaagtcttgcttagttccttcgtgcagcttcttgcccctgttgacgtggaatgctgtgtctgctttagcacgcacgctccgaatgactcctggtgctaggccatgctggctgctgtcactgagcgggactcaggccaagaggcgtgacctcgggccagcctgtctgttgtgcagacgcctcctctgcagaacgcatcagtttctattctgcagttgcagagccagccccgcgtgagaacgtgcataatgagtgcacaccatcatgtcaaggtgcatacttagtgagcgccatcctgctgaacgtgtatttcagtgtttcacttactggacggataacaagaaaaaaatcctaacacaggcagtcaccagaaataaatgtctcagcactttacagatgactaaaaatgttaattttatgacttagccaaatatgttctaggttgcatatatcccccatgtgaaagtgatttcttcccaagcttctcaaactgttagctgctgtctgacttcatcaataaagtatttttattttaaaatgcaaaaaaaaaaaaaaaaaaaaaaaaaaaaaaaaaaaaa

SEQ ID NO: 2 - protein, 1306 aa, Homo sapiens [sequence omitted]

SEQ ID NO: 3 - DNA, 3549 bp, Caenorhabditis elegans
  CDS (1)..(3549)
atgctcaacccaaacgatgcagacgattcggatagttcgactgactcttcaccgcccgttgccgtgccgagacccaagcgttcggctaccgtatcacccagcactagaaattcattttataattccagaagacggagtgatgcaaatctaaactttttcaacgagagagaacgccagcgtgagagacgcagaagcatggttcgtgcgagccgcgagagaagtgaatccagaagagcttcacaacaaaatctcaggaaaacgaatagtgagccaaatgtggatatgccatcgatcgatgtggaagctcttcaaaagctattactttccattccgaaatttgtgtcggcttccagaaatcgaataaaccaacggcacgattcggattccggtggagaaatgacaactgagatcgaagaactcaaagatgccgcaaaaagtattcaatccctacaacgtgtgctcgcctatccatcccaaacatcttcggatcgtggaggtaaccgtcgtctagcagatcccgtcgagtcgtgccttgaatcttcagttttctccgacgtcgacgagtcctcgctggccactgacaccatcgaaggtcgacgatctgggatctcaactcgactgggtcatccacgaggtgttatgcaatttattccatctataagaaacgatcaatttgtgcctacttccgagatacggaggaactcgatttccagaaaatcttcggtacccgatgggtttatgactagtgaaatggagtccgagccggtaccaaatgttaggaatagacgccgtggaatagtcgatgaaatgttctcctcttcaacatctctcctcgtggatagaccgtctgaaacccttgcaggagtgaatcggtttgcaaagcttcttgatacttttcgcagtcgacccacatccccggagcagcacccttcaatctcgtggaatccatatgtttattctgatggaggggaagctctagagacctgtgggatggaagattctttacacgaagctgacattttgttatggaagaagaggagtcgggcaagtctacgaagacattatagtgttagacaccttgctgctcgagagctgttagacactgagaagtcatttgtagagggcctagagttcctggttacgaagtacatgagacctctccgtcaaccattagaatgcacactgatcgaagcatcactggtagacaagattttctaccggataccagagattctggctcatcaccaagttttgctcactacgctgagccaacggatagatcagtggcataaagatgcaatactgggagatgtgcttctagcccatttctccaagcaatcaatgatagaaacctatatctcatttgtggataatttcaagtttgccaaggcatcaataatccaagccagacagaagcacgcatttgaaaaatattataatagatgctgccgagaccatcctaacaagttggacctagattctcttcttatttcaccgattcaacgcgttcctagatacgaattaatagtaaaacaaatgctgaaacacactcccgttgaacacgaggatcgagagcgactgctcagggctcagaggcatattcattgtcttgctgtggcaatcaatcagcataaggatggaagtgagcaaatggaacagagactaagggaaattgaggccatagttgatgggttggatgatctcgtcaccaaagatcggacccttctacggcatgacatcattacactgaaggggaccgatcgagagcggtgtatatttatgctatctgatcttttactggttacgagtgtcaagaagaagcccaaggtcttgtatagtaaaatgtcctcccaatccatgggcttcttggagggaaatcgtttcaagctcctgttcaaagtggcactggaagatgttcaaatatcaaaagacacactttcacaactagaagaagttgagcggaaactggagtcctctcgggaagatgatcgagttcttaagaaaatgtcgcaattgtgcagtctgttgaagtgcgaaaggaaggttctaatggagatgttggagaccatggagacctctaattcaatgagcattcgagagctcaacgagcaaatgagctccgatccagatttgagtgctgttcatttggatgttctcacctcgaacggattcgagccatttgtgttggagtttccaaatgctgaaaagcgaagtgtgtttgaagcgatgtttaaggatgcgaaggcaacgttagcaaagaatttgttggctgcaccgagctgtagtctaaaaacaattattgctcatcagacaaggcccgggttgcagctctgtaccgccacagtagtccctggaaaacgtgtcgacagtaccccatcactttgggtttgtgctagcgataagttcagtggacaggtagccgtaatggcattggataccggagaaataactattgaaagctgctcggcaatcggtaatgcagcggtgactgcgatgtgtactgtacccccgcctatgaaacttcgaaaaagaaaaattaaatctcaaaagtctctggagcatctgctaaatgagacaataatggatataaatagtagtggctcggatacagaatcctcttccgatgagggaacatcaactgcaggacagacaacagtatggattggaaatgatgatggagaagtgttcgtggttaactccacagagcgagtgaggtcccgggcccgagatcgactagctcgacttcggaatagtatcacatcaatctgtgcggcaaacgggaatgtactagttgccacgtcatattcgaatcaagtacagcttcttctatttcgtccggcatcggatggaagttgggatttagagaatccacaaacagttggacatgtatgtcaggcaccgatcacgtcgatgcagttgattggtagaagggtaattattgctagtggcaattggttacacgcgtattttgtggataccgggaagtttcagccaccggtggaaatcctcccctcttcggatgtgatcaccctaatgtatgttaccggacaaaatgtattcctatgcggcagaaaatcaaccgaagtctttgtagttgatgttttcaatttatcgattatcaatcatttcaatgttgtttcatttgtccgttcacaactttccggtagagagcacatcctccgtgagcacaaaatgggatgccttcggatatcgtgtttgacagttgctagatctcatctttggattggcacatctgctggatgtgtgctatcaacttctgtacaatctgcgagatctcaaccgacacccgatcttcgtgtctgcgaaatcggccacagtggaccttgtcgaatccttctgccagttcatacaccgtctcactcaaaccatccttcaagaaaacagaagcgaagttctttaaatgttccagcacaacaatcatcacagctaatgttggtcagttgcggcgagggtctcgacgacggaacggccactcaagacccatcaacggatgccataaatcatttgatcttttggaaatgctcataa

SEQ ID NO: 4 - protein, 1182 aa, Caenorhabditis elegans
MLNPNDADDSDSSTDSSPPVAVPRPKRSATVSPSTRNSFYNSRRRSDANLNFFNERERQRERRRSMVRASRERSESRRASQQNLRKTNSEPNVDMPSIDVEALQKLLLSIPKFVSASRNRINQRHDSDSGGEMTTEIEELKDAAKSIQSLQRVLAYPSQTSSDRGGNRRLADPVESCLESSVFSDVDESSLATDTIEGRRSGISTRLGHPRGVMQFIPSIRNDQFVPTSEIRRNSISRKSSVPDGFMTSEMESEPVPNVRNRRRGIVDEMFSSSTSLLVDRPSETLAGVNRFAKLLDTFRSRPTSPEQHPSISWNPYVYSDGGEALETCGMEDSLHEADILLWKKRSRASLRRHYSVRHLAARELLDTEKSFVEGLEFLVTKYMRPLRQPLECTLIEASLVDKIFYRIPEILAHHQVLLTTLSQRIDQWHKDAILGDVLLAHFSKQSMIETYISFVDNFKFAKASIIQARQKHAFEKYYNRCCRDHPNKLDLDSLLISPIQRVPRYELIVKQMLKHTPVEHEDRERLLRAQRHIHCLAVAINQHKDGSEQMEQRLREIEAIVDGLDDLVTKDRTLLRHDIITLKGTDRERCIFMLSDLLLVTSVKKKPKVLYSKMSSQSMGFLEGNRFKLLFKVALEDVQISKDTLSQLEEVERKLESSREDDRVLKKMSQLCSLLKCERKVLMEMLETMETSNSMSIRELNEQMSSDPDLSAVHLDVLTSNGFEPFVLEFPNAEKRSVFEAMFKDAKATLAKNLLAAPSCSLKTIIAHQTRPGLQLCTATVVPGKRVDSTPSLWVCASDKFSGQVAVMALDTGEITIESCSAIGNAAVTAMCTVPPPMKLRKRKIKSQKSLEHLLNETIMDINSSGSDTESSSDEGTSTAGQTTVWIGNDDGEVFVVNSTERVRSRARDRLARLRNSITSICAANGNVLVATSYSNQVQLLLFRPASDGSWDLENPQTVGHVCQAPITSMQLIGRRVIIASGNWLHAYFVDTGKFQPPVEILPSSDVITLMYVTGQNVFLCGRKSTEVFVVDVFNLSIINHFNVVSFVRSQLSGREHILREHKMGCLRISCLTVARSHLWIGTSAGCVLSTSVQSARSQPTPDLRVCEIGHSGPCRILLPVHTPSHSNHPSRKQKRSSLNVPAQQSSQLMLVSCGEGLDDGTATQDPSTDAINHLIFWKCS

SEQ ID NO: 5 - DNA, 2485 bp, Caenorhabditis elegans
cgtagaaattgcccggcgaatcttgtcttcaatccagcgatttcctcttgtgattggccaaaaaatgtgatggattgttctgaaaaatctgaaaagccacaaaactgcggcgaagtcgacggatatttctcatttggcagatgctcttcatcattttcagcctgtacaaatggcattccaattgtcatgttttgtccagatgggctaatgttttccgagaaaaatcaaatgtgtgactatgaatggaacgttgacgagtgtgacttggaatcatctggatttatggaaaactacaaagccagtgaagccttaacgccctgcacaaatatggacaatggtctctatgctctcgattgtaccccgcgagttctatcgtgtcaaaatggacgagagaatatttttgagtgcccaccgagtctggttttcaatgagaattctttgatatgtgactacccggagacttctctgaaatgctgcatggaaggtaatagtgattccaaattacaattgaataaaacgatttttcagacgcattgctcattcgtgatgcctccattggaacatacgactgctccatcgacgggctcttctcaagtactttatgctcccgtaactaccacaaatgctccaacggccagctcatcccacacgaatgtgctgattcgaatgctgttttctctgcaagtgctgccaaatgcgtggattcttcacttctagagcaatgccaataatctgattatagctgtatatactaaccctttgctgaaaaatatattgcattttaaattttgtcttgaaaatattttttaaattgtgaattttatacaaaaataaatctttgctgttttcatacgaaatgtccacaaaaattaaaaaaaaaacataccaagggaatttttctgagttgattcaaaattgaaaaaaggtatcgggccgttcaagtttcaatttttcaagaaatgttggctgtagttcaaaattaattgatgcgaaacggatgaaactaattgaaaacatgaatttgcagttttggcgccaaataccttacgagacccatcgttctgaggcggggctttttcaattgacaatggagcgcgttttcacgttgacttgaaaaatgaatttttctgcctacctataataattttttcaggaaaatttgaaagaaaattcaattttgaatgtttctaaaaaaagttggatacaaatttgagagtcattaattttaccagatcattgtcataaccaatcataattgttttccctctcacccttatccactaaataaaaaccaattgttcacaaagttgaccttatcccttcttcctcttcacatgagtgatgagcaagaaggcaggtaggcttctccagacgggaacggcctcttcttatccgtcagctgacatcttactctacatcaagttgtggaggacatctctacgcattgattggtcacttttgctcattggccttcttccaccacccccgtcatctgaaaatattgtttttgattagttgttgggaagaatagtatattgttttattgatctcgtgtcgtcatatgcttttttcgattataaatttttagagcttgtaatagtttttctttgcttctcagtcaaaatgctttatggattttttttaaaaaagagcaaaaaccaaaacgatcaacacaaaaacaattgtaattaaaaattctctttaaaacattttcacttcctatttttgatagttaacttttttttatgaatttcacatttttagagtttcaattttcggttttcctataactacaacatttcaacattttttttggaaatgatgttttaatttactattcaaaagtaggtaaaactctttgtagttgctgttgaattctagataaaacaagaatttgtaggctttaagaaataaatacatattttttcaaactgtaaatacacaaaaacactgataatttgaatgcaaaaaataaaaagaattgtgaaaaatagttcaaaatcagatatttttgcccatagctcttagaatcctcggagagtacatgagctgggtaagttgacacttgaatatataaagggctaattatttggtagaatgacgtcacacaccattttctgtaggcttttacagctggatgtgacgtcacataaaggaaaagccaagtgatttctctccgccctaaactaagtgaagaataagttcctagacaccttggaggtcccatctttctctctacttctctcgcctcacaacattgctctcaataacacaacaaagagggaatcagttacacctgagagcaatagaatgctccaattgaaagttcaaaatgctcttttgtttcctaccttcaaaacgccatatgactttaagctttttttcattctttttgcagaggagcaaaggtttcaagctcaaattccctgttatctgggggtctcttgagtctccattctatatcattggctcccctgtggctcaa

SEQ ID NO: 6 - DNA, 5034 bp, Artificial Sequence
  myo-2 promoter sequence operably linked to a GFP coding sequence
aagcttgcatgcctgcaggtcgaggcatttgaattgggggtggtggacagtaactgtctgtaataataattactcctgaccaggttgcaattcgagttttgataagcataattataccttgtacattgtgggttttgtgctgtggacgttttattgtgacatccccataagctacaagaacaaaaatgaaatgtaaaagtattgaaaacgtcgtacattttatatctgagtagtatcctttgctttaaatgtccataaaactaattttataatcaataaaacaacgtttgtaaatcaactgagtttacaagtagagacattgagggatactttcactatgctaaagtgataatcgaccaaataataacttcactttggtatttattcctgtcttataaatgttatgtatgaattaaattcatatgcatatggctcactctgacaatttttaataatcttccagatcaatattgactaccgatgcgggtggtcttttgctttgaattctgctgaactttacaccccgaacagcaatgtgtgcttcagcctaaaaaaaagtaagtgtgttaatcagtgcccccgattcttcattttttgcccctctctcccgtttcgtcggcaaaagaagagaaaataaagataagtctcaagataggttggtaatcgctaaagtggttgtgtggataagagtagcaaaatggcaggaagagcactttgcgcgcacacactgtactcattgttctggataaaattctctcgttgtttgccgtcggatgtctgcctctctgcattgagccggcttcttcactatctttagttaacctaaaatgccgtttcttttctcgtatccccactatcccgttgaggttctctgctctcttcgctccctaccgccagcgagcaactatccgtgggggcgccttgctcggaagatgggggggaagaaagaagatttttgctatttgcacttgagaaagagacttttcctgcgtcgatggttagagaacagtgtgcagacacttttcagctacctagaattacaattggatatccccgcctcccaatccacccacccagggaaaaagaagggctcgccgaaaatcaaagttatctccaggctcgcgcatcccaccgagcggttgacttctctccaccacttttcattttaaccctcggggtacgggattggccaaaggacccaaaggtatgtttcgaatgatactaacataacatagaacattttcaggaggacccttggctagcgtcgacggtaccggtcagatatcactcgaggagctcccgagatcctatcgattcgcggccgctgtacacccgggtgccctatagtgagtcgtattggtaccggtagaaaaaatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatgggtaagtttaaacatatatatactaactaaccctgattatttaaattttcagccaacacttgtcactactttctgttatggtgttcaatgcttctcgagatacccagatcatatgaaacagcatgactttttcaagagtgccatgcccgaaggttatgtacaggaaagaactatatttttcaaagatgacgggaactacaagacacgtaagtttaaacagttcggtactaactaaccatacatatttaaattttcaggtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttgtaagtttaaacatgattttactaactaactaatctgatttaaattttcagaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggacgaactatacaaaagcccacaagctttggaattcgctagccggccatacaagtaatccggatgatcgacgccaacgtcgttgaattttcaaattttaaatactgaatatttgttttttttcctattatttatttattctctttgtgttttttttcttgctttctaaaaaattaattcaatccaaatctaaacatttttttttctctttccgtctcccaattcgtattccgctcctctcatctgaacacaatgtgcaagtttatttatcttctcgctttcatttcattaggacgtggggggaattggtggaagggggaaacacacaaaaggatgatggaaatgaaataaggacacacaatatgcaacaacattcaattcagaaatatggaggaaggtttaaaagaaaacataaaaatatatagaggaggaaggaaaactagtaaaaaataagcaaagaaattaggcgaacgatgagaattgtcctcgcttgggcccaaaaggccttttttttttggcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgtaagtttaaacatgatcttactaactaactattctcatttaaattttcagagcttaaaaatggctgaaatcactcacaacgatggatacgctaacaacttggaaatgaaat

SEQ ID NO: 7 - DNA, 28 bp, Artificial Sequence
  forward primer
aactgcagtgtgacttgggatcatcgga

SEQ ID NO: 8 - DNA, 30 bp, Artificial Sequence
  reverse primer
cgggatccttgagccacaggggagccaatg